SEQUENCE LISTING (7 sequences)

SEQ ID NO: 1 - protein, 35 aa, Artificial
  Channel blocker GsMTx-4 (Grammostola spatulata venom 4 kDapeptide)
GCLEFWWKCNPNDDKCCRPKLKCSKLFKLCNFSSG

SEQ ID NO: 2 - protein, 759 aa, Homo sapiens
MMAALYPSTDLSGASSSSLPSSPSSSSPNEVMALKDVREVKEENTLNEKLFLLACDKGDYYMVKKILEENSSGDLNINCVDVLGRNAVTITIENENLDILQLLLDYGCQKLMERIQNPEYSTTMDVAPVILAAHRNNYEILTMLLKQDVSLPKPHAVGCECTLCSAKNKKDSLRHSRFRLDIYRCLASPALIMLTEEDPILRAFELSADLKELSLVEVEFRNDYEELARQCKMFAKDLLAQARNSRELEVILNHTSSDEPLDKRGLLEERMNLSRLKLAIKYNQKEFVSQSNCQQFLNTVWFGQMSGYRRKPTCKKIMTVLTVGIFWPVLSLCYLIAPKSQFGRIIHTPFMKFIIHGASYFTFLLLLNLYSLVYNEDKKNTMGPALERIDYLLILWIIGMIWSDIKRLWYEGLEDFLEESRNQLSFVMNSLYLATFALKVVAHNKFHDFADRKDWDAFHPTLVAEGLFAFANVLSYLRLFFMYTTSSILGPLQISMGQMLQDFGKFLGMFLLVLFSFTIGLTQLYDKGYTSKEQKDCVGIFCEQQSNDTFHSFIGTCFALFWYIFSLAHVAIFVTRFSYGEELQSFVGAVIVGTYNVVVVIVLTKLLVAMLHKSFQLIANHEDKEWKFARAKLWLSYFDDKCTLPPPFNIIPSPKTICYMISSLSKWICSHTSKGKVKRQNSLKEWRNLKQKRDENYQKVMCCLVHRYLTSMRQKMQSTDQATVENLNELRQDLSKFRNEIRDLLGFRTSKYAMFYPRN

SEQ ID NO: 3 - DNA, 4085 bp, Homo sapiens
ccgggcctcgagccgaggcagcagtgggaacgactcatcctttttccagccctggggcgtggctggggtcggggtcggggtcggggccggtgggggccccgcccccgtctcctggcctgcccccttcatgggccgcgatgatggcggccctgtacccgagcacggacctctcgggcgcctcctcctcctccctgccttcctctccatcctcttcctcgccgaacgaggtgatggcgctgaaggatgtgcgggaggtgaaggaggagaatacgctgaatgagaagcttttcttgctggcgtgcgacaagggtgactattatatggttaaaaagattttggaggaaaacagttcaggtgacttgaacataaattgcgtagatgtgcttgggagaaatgctgttaccataactattgaaaacgaaaacttggatatactgcagcttcttttggactacggttgtcagaaactaatggaacgaattcagaatcctgagtattcaacaactatggatgttgcacctgtcattttagctgctcatcgtaacaactatgaaattcttacaatgctcttaaaacaggatgtatctctacccaagccccatgcagttggctgtgaatgcacattgtgttctgcaaaaaacaaaaaggatagcctccggcattccaggtttcgtcttgatatatatcgatgtttggccagtccagctctaataatgttaacagaggaggatccaattctgagagcatttgaacttagtgctgatttaaaagaactaagtcttgtggaggtggaattcaggaatgattatgaggaactagcccggcaatgtaaaatgtttgctaaggatttacttgcacaagcccggaattctcgtgaattggaagttattctaaaccatacgtctagtgacgagcctcttgacaaacggggattattagaagaaagaatgaatttaagtcgtctaaaacttgctatcaaatataaccagaaagagtttgtctcccagtctaactgccagcagttcctgaacactgtttggtttggacagatgtcrggttaccgacgcaagcccacctgtaagaagataatgactgttttgacagtaggcatcttttggccagttttgtcactttgttatttgatagctcccaaatctcagtttggcagaatcattcacacaccttttatgaaatttatcattcatggagcatcatatttcacatttctgctgttgcttaatctatactctcttgtctacaatgaggataagaaaaacacaatggggccagcccttgaaagaatagactatcttcttattctgtggattattgggatgatttggtcagacattaaaagactctggtatgaagggttggaagactttttagaagaatctcgtaatcaactcagttttgtcatgaattctctttatttggcaacctttgccctcaaagtggttgctcacaacaagtttcatgattttgctgatcggaaggattgggatgcattccatcctacactggtggcagaagggctttttgcatttgcaaatgttctaagttatcttcgtctcttttttatgtatacaaccagctctatcttgggtccattacagatttcaatgggacagatgttacaagattttggaaaatttcttgggatgtttcttcttgttttgttttctttcacaattggactgacacaactgtatgataaaggatatacttcaaaggagcagaaggactgtgtaggcatcttctgtgaacagcaaagcaatgataccttccattcgttcattggcacctgctttgctttgttctggtatattttctccttagcgcatgtggcaatctttgtcacaagatttagctatggagaagaactgcagtcctttgtgggagctgtcattgttggtacatacaatgtcgtggttgtgattgtgcttaccaaactgctggtggcaatgcttcataaaagctttcagttgatagcaaatcatgaagacaaagaatggaagtttgctcgagcaaaattatggcttagctactttgatgacaaatgtacgttacctccacctttcaacatcattccctcaccaaagactatctgctatatgattagtagcctcagtaagtggatttgctctcatacatcaaaaggcaaggtcaaacggcaaaacagtttaaaggaatggagaaatttgaaacagaagagagatgaaaactatcaaaaagtgatgtgctgcctagtgcatcgttacttgacttccatgagacagaagatgcaaagtacagatcaggcaactgtggaaaatctaaacgaactgcgccaagatctgtcaaaattccgaaatgaaataagggatttacttggctttcggacttctaaatatgctatgttttatccaagaaattaaccattttctaaatcatggagcgaataattttcaataacagatccaaaagactatattgcataacttgcaatgaaattaatgagatatatattgaaataaagaattatgtaaaagccattctttaaaatatttatagcataaatatatgttatgtaaagtgtgtatatagaattagttttttaaaccttctgttagtggctttttgcagaagcaaaacagattaagtagatagattttgttagcatgctgcttggttttcttacttagtgctttaaaatgtttttttttatgtttaagaggggcagttataaatggacacattgcccagaatgttttgtaaaatgaagaccagcaaatgtaggctgatctccttcacaggatacacttgaaatatagaagttatgttttaaatatctctgttttaggagttcacatatagttcagcatttattgtttaggagtataattttattttatctaaaataatagtctattttttcttttgtattttgttataatcttaagcaacaaagaaaaaaccctaatatttgaatctatttatgtctttcaatttaaattcacttcagtttttgttattgtaatatatttacttttacatggttataatcactttatatttttaatgtttttttcacttaatattttatatatacatttccatgtattgatgtagttagtccacatttaaatttttatagaattatatagtttttgaaaaatacagtcagtagatgttttattttttagctattcagttatgtttataagtttgcatagctacttctcgacatttggtttgttttaatttttttgtatcataatagtcctatttttttttcaagttggagtgaatgtttttagttttaagatagataggagacacttttttatcacatgtagtcacaacctgttttgtttttgtaaaacataggaagtctctttaatgcaatgatttgttttatatttggactaaggttcttgagcttatctcccaaggtactttccataatttaacacagcttctataaaagtgacttcatgcttacttgtggatcattcttgctgcttaagatgaaaagcattggttttttaaaattagagaataaaatatgtatttaaatttttggtgtgttcacataaagggatgtagctaaaatgttttcataggctattatatattctcgcagcatttccagttaagaggatattaggtatataattctcttcttaaccgaatgtcagatggtcttacgccacagggtgcaggtaacccttggtctgtaagcaccaccgatccagggatcattgtctaaataggttactattgtttgtttcatcttgcttttgcatttttattttttaatttccaaattttaagtgttccctctttggggcaaattcttataaaaatgtttattgtaaagttatatattttgtctacgatgggattatgcacttcccaattgggattttacatctggatttttagtcattctaaaaaacacctaattattaaaacatttatagagtgcctactgtatgcatgagttgagttgcttctgaggtacattttgaatgacagcatattgtaagaaaaaaaaaggtgaataaaatttgacattagattataaaaaaaaaaaggaattc

SEQ ID NO: 4 - DNA, 30 bp, Artificial
  Scrambled antisense oligonucleotide
cttttgaccgctcatccctatagtatttgc

SEQ ID NO: 5 - protein, 12 aa, Artificial
  Epitope of MscCa polypeptide
CVGIFCEQQSND

SEQ ID NO: 6 - protein, 15 aa, Artificial
  Epitope of MscCa polypeptide
QLYDKGYTSKEQKDC

SEQ ID NO: 7 - protein, 31 aa, Artificial
  Channel blocker GsMTx-1
YCQKWMWTCDEERKCCEGLVCRLWCKKKIEW

(19) United States
(12) Patent Application Publication
(10) Pub. No.: US 2007/0190600 A1
(43) Pub. Date: Aug. 16, 2007

(54) Mechanosensitive ion channels and methods of use

(75) Inventors: Owen P. Hamill, Galveston, TX (US); Rosario Marato, Galveston, TX (US)

Correspondence Address: MUETING, RAASCH & GEBHARDT, P.A., P.O. BOX 581415, MINNEAPOLIS, MN, 55458, US

(73) Assignee: Board of Regents, The University of Texas System, Austin, TX (US)

(21) Appl. No.: 10/585,503

(22) PCT Filed: Jan. 10, 2005
(86) PCT No.: PCT/US05/00722

§ 371 Date: Jan. 17, 2007

Related U.S. Application Data
(60) Provisional application No. 60/535,327, filed on Jan. 9, 2004.

(51) Int. Cl. C07K 14/705; C07H 21/04; C12P 21/06

(52) U.S. Cl. 435/69.1; 435/320.1; 435/325; 530/350; 536/23.5

(57) ABSTRACT

The present invention provides methods for identifying agents that decrease the activity of a mechanosenitive ion channels, preferably, a mechanosensitive Ca²⁺-permeable channel (MscCa) channel. The present invention also provides methods for using agents that decrease the activity of mechanosenitive ion channels, including, for instance, methods for treating cancer, methods for decreasing metastasis of a cancer cell, and methods for decreasing a symptom associated with cancer.

### CONTINUING APPLICATION DATA

This application claims the benefit of U.S. Provisional Application Serial No. 60/535,327, filed Jan. 9, 2004, which is incorporated by reference herein.

### BACKGROUND

Cancer is a disease in which abnormal cells divide without control. Cancer cells can invade nearby tissues and can spread through the bloodstream and lymphatic system to other parts of the body. More than 1.2 million Americans develop cancer each year, and it is the second leading cause of death in the United States. There are several types of cancer, defined by their tissue of origin. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the bloodstream. Lymphoma is a cancer that begins in the cells of the immune system. Cancerous cells destroy the part of the body in which they originate, and some may spread to other parts of the body where they cause more destruction.

Prostate cancer, a carcinoma, is the most common cancer other than superficial skin cancer, and is the second leading cause of cancer death in American men. Furthermore, between 1976 and 1994, prostate cancer rates doubled and mortality increased by 20% (Haas G. & Sakr W., CA Cancer J. Clin., 47, 273-287 (1997)). The reasons for the increase are not known, but increasing life expectancy, growing disease prevalence resulting from environmental carcinogens, and increasing use of novel diagnostic modalities have been suggested as causes. Most prostate cancers are slowly progressive malignancies, and many are present for years before they are identified by clinical diagnosis. In the early stages, the disease stays in the prostate and is not life threatening, but without treatment it metastasizes to other parts of the body and eventually causes death. Current therapies are limited to treatment within the prostate, and there is no cure once it has spread. Therefore, identifying methods of preventing the metastasis of prostate cancer are crucial for treatment of the disease:

The process of cancer metastasis consists of a long series of sequential, interrelated steps, each of which is rate limiting. After the initial transforming event, growth of neoplastic cells is progressive. Extensive vascularization (angiogenesis) must occur if a tumor mass is to exceed 2 mm in diameter. The synthesis and secretion of several angiogenic factors play a key role in establishing a capillary network from the surrounding host tissue. Invasion of the host stroma by some tumor cells occurs by several non-mutually exclusive mechanisms (Liotta LA, Cancer Res., 46, 1-7 (1986)). Thin-walled venules, like lymphatic channels, are easily penetrated by tumor cells and provide the most common pathways for tumor cell entry into circulation. Detachment and embolization of small tumor cell aggregates occurs next, and the majority of circulating tumor cells are destroyed rapidly. Tumor cells that survive circulation must be trapped in the capillary beds of organs. Extravasation follows next, by the same mechanisms that influenced initial invasion, and the development of vascularization and proliferation within the organ parenchyma completes the metastatic process. Tumor cells can invade host stroma, penetrate blood vessels, and enter the circulation to produce additional metastases, the so-called “metastasis of the metastases.” In fact, cells that populate metastases have been shown to possess a greater metastatic capacity than most cells in the parent neoplasm (Talmadge J. & Fidler I., J. Natl. Cancer. Inst., 69, 975-980 (1982)).
It is now recognized that a tumor cell's ability to migrate plays a critical role in the spread of prostate cancer (Banyard, J. and Zetter, B.R., Cancer and Metastasis Reviews, 17, 449-458 (1999)j. In order for prostate cancer to metastasize, a tumor cell must migrate from the prostate, pass through blood vessels, penetrate the secondary tumor site, and migrate through the tissue to establish a metastatic site. Metastasis will only occur if the tumor cell completes every step in this cascade. Thus, identifying a dysregulated step in the transformation of non-motile into motile tumor cells should provide a promising therapeutic target for preventing prostate tumor cell spread and metastasis. A number of researchers have been investigating potential rate-limiting processes in cell motility (Sheetz et al., Biochem. Soc. Symp. 65, 233-243 (1999); Kassis et al., Cancer Biology 11, 105-117 (2001); Wells et al., Acta Oncologica, 41, 124-130 (2002)).
Cell motility plays a role in a variety of physiological processes including embryogenesis, inflammation and wound healing. Although motile cells vary in the exact details and rates of their movement (ranging from <I mm/minute for fibroblasts and tumor cells to approximately 30 minute for fish epithelial keratocytes), they appear to share common features (Lauffenburger & Horwitz, Cell, 84, 359-369 (1996). For example, the traction forces exerted by motile cells are transmitted to the underlying substrate through nascent focal adhesions most likely formed by integrins linked to the actin cytoskeleton. Furthermore, cell movement depends upon the ability of the focal adhesions to assemble and disassemble in response to changes in locally applied force. Therefore, a question in cell motility concerns the nature of the mechanosensitive processes that regulate the assembly and disassembly of focal adhesions. In several migrating cells, spatial and temporal gradients in intracellular Ca²⁺concentration ([Ca²+₁]) have been demonstrated, with lower [Ca²+]i in the front that would favor adhesion assembly, and higher [Ca²+)] in the rear that would promote adhesion disassembly and release (Schwab, A., Am. J. Physiol. 280, F739-F747 (2001)). Based on inhibition studies, several Ca²+-dependent molecules have been implicated as participating in adhesion disassembly, including calcineurin, a Ca 2+/calmodulin-activated phosphatase, and calpain, a Ca²+-activated cysteine protease (Geiger, B. and Bershadsky, A., Curr. Opin. Cell Biol. 13, 584-592 (2001); Webb et al., Nature Cell Biol., 4, E97-EI 00 (2002)).
Mechanosensitive ion channels (MSCs) were first discovered in tissue cultured skeletal muscle cells using single channel patch clamp recordings, and have since been found in both the plant and animal kingdoms and in the cells of most tissues, including myocardial tissue. Most of them open with increasing membrane tension (stretch-activated channels (SACs)), but a few are tonically active and close with increasing tension (stretch-inactivated channels (SICs)). The ion selectivity of the MSC channel family is variable, and as such is similar to that seen for voltage-activated or ligand-activated channel families. In the animal cells, the most common forms are cation selective and, more particularly, potassium selective. The cation channels will pass divalent ions such as Ca+² and Ba+² as well as monovalent ions. MSCs have been implicated as either activators or modifiers of many different cellular responses to mechanical stimuli including modification of electrical and contractile activity of muscle tissue. Consequently, MSCs have been primarily studied for their involvement in the mechanical sensitivity of the heart. Mechanical stimulation of cardiac myocytes and whole heart preparations can cause depolarization, extrasystoles and arrhythmias (Hu et al., J. Mol. Cell Cardiol., 29:1511-1523 (1997)).
Studies in various cell types indicate that MSC activity may be involved in cell motility. For example, in the highly motile fish epithelial keratocyte, Lee et al., Nature, 400, 382-386 (1999), have used Ca²+imaging and patch-clamp recording to demonstrate that transient increases in [Ca 2+] coincide with phases of increased mechanosensitive Ca²+-permeable channel (MscCa) activation associated with cell extension. MscCa belong to the class of mechanosensitive channels (Sachs & Morris, Revs Physiol. Biochem. & Pharm. 132, 1-77 (1998); Hamill & Martinac, Physiol Revs., 81, 686-740 (2001)), and transduce membrane stretch into Ca +influx, thereby providing positive feedback between cell extension and Ca²+-dependent disassembly mechanisms. Studies using gadolinium ion (Gd³+), a non-specific channel blocker, indicated that Gd³+abolishes Ca²+transients and prevents rear detachment, thereby inhibiting cell migration (Lee et al., Nature, 400, 382-386 (1999)). However, determining the role of MscCa in cell motility is complicated by the fact that changes in its expression, or coupling with a downstream Ca²+signaling mechanism(s), could also be rate-limiting.
Other types of ion channels, including voltage-gated Na+channels (VGSC) and store-operated Ca²+channels (SOCC), have been studied in prostate tumor cells (Diss et al., FEBS Letts., 427, 5-10 (1998), Gutierrez, et al., J. Physiol. 517.1: 95-107 (1999)). For example, enhanced VGSC expression has been correlated with increased tumor cell invasiveness, while tetrodotoxin, a VGSC blocker, has been shown to reduce invasiveness (Grimes, et al., FEBS letts. 369, 290-294 (1995); Smith, et al., FEBS Letts. 423, 19-24, (1998)). Invasiveness, with respect to these studies, was measured in vitro by the ability of tumor cells to actively migrate through a basement membrane matrix (e.g., Mairigel), and also depends upon adhesion receptors for matrix attachment and metalloproteinases for matrix lysis. It is unclear at this stage how the VGSC is activated in prostate tumor cells. One interesting possibility is that it occurs via an MscCa-induced membrane depolarization. However, a pharmacological study attempting to address the role of MscCa reported that Gd³+enhanced rather than inhibited prostate tumor cell motility (Vera)l et al., Cancer Letis., 145, 79-83 (1999)). Although this result appears to question the generality of an earlier epithelial keratocyte result (Lee et al., Nature, 400, 382-386 (1999)), the effects of Gd³+on prostate cell motility were unfortunately measured in a medium containing serum, which includes components that avidly bind Gd³+and reduces its blocking efficacy (HamilJ & McBride, Pharmacol. Rev., 48, 231-252 (1996); Caldwell, et al., Am. J. Physiol. 275, C619-C621 (1998)). Furthermore, apart from being a nonspecific channel blocker, Gd 3+has also been reported in l5 some cells to have concentration-dependent biphasic (stimulatory and inhibitory) effects on mechanosensitive channels (Hamill & McBride, Pharmacol. Rev., 48, 231-252 (1996)). These concerns indicate the necessity of directly measuring Gd³+effects on both MscCa and prostate tumor cell migration under the same conditions.

### SUMMARY OF THE INVENTION

The invention provides a method for identifying an agent that decreases activity of a mechanosensitive Ca²+-permeable (MscCa) channel that includes the steps of contacting a cell expressing an MscCa channel with a candidate agent to yield a treated cell and comparing the activity of an MscCa channel of the treated cell with the activity of an MscCa channel of a control cell not contacted with the candidate agent. The decreased activity of an MscCa channel of the treated cell then indicates the candidate agent decreases the activity of an MscCa channel. The method for identifying an agent may include an MscCa channel that is a polypeptide including an amino acid sequence of at least 90% identity to SEQ ID NO: 2 that retains MscCa activity. More particularly, the MscCa channel may be a polypeptide including SEQ ID NO: 2. The cell expressing an MscCa channel used in the method for identifying an agent may be a tumor cell. An embodiment of the method may include cells of a human prostate tumor cell line. This embodiment may further include human prostate tumor cells of the ATC CRL-1435 line.
The method for identifying an agent may include a cell expressing an MscCa channel that is a motile cell. In this embodiment of the invention, the treated motile cell has a decreased motility compared to the control cell. In a further embodiment of the method, the cell is an invasive cell, and the treated invasive cell has decreased invasiveness compared to the control cell. In an additional embodiment, the treated cell has decreased proliferation compared to the control cell. An in yet another embodiment, the treated cell has increased apoptosis compared to the control cell.

The invention also includes an agent identified by a method for identifying an agent that decreases activity of a mechanosensitive Ca²⁺-permeable (MscCa) channel that includes the steps of contacting a cell expressing an MscCa channel with a candidate agent to yield a treated cell and comparing the activity of an MscCa channel of the treated cell with the activity of an MscCa channel of a control cell not contacted with the candidate agent, in which the decreased activity of an MscCa channel of the treated cell then indicates the candidate agent decreases the activity of an MscCa channel.

The invention also includes a method for identifying an agent that decreases a phenotype of a cell including the steps of contacting a cell expressing an MscCa channel with a candidate agent to yield a treated cell and comparing the phenotype of the treated cell with the phenotype of a control cell not contacted with the candidate agent. In the method, the phenotype is selected from the group of motility, invasiveness, proliferation, or a combination thereof, and a decreased phenotype for the treated cell indicates the candidate agent decreases the phenotype. In an embodiment of the method, the candidate agent causes the activity of an MscCa channel of the treated cell to decrease. The MscCa channel in the method may be a polypeptide including an amino acid sequence of at least 90% identity to SEQ ID NO: 2 that retains MscCa activity. More particularly, the MscCa channel may be a polypeptide including SEQ ID) NO: 2.

The cell expressing an MscCa channel used in the method for identifying an agent that decreases a phenotype of a cell may be a tumor cell. An embodiment of the method may include cells of a human prostate tumor cell line. This embodiment may further include human prostate tumor cells of the ATC CRL-1435 line.

The invention also includes an agent identified by a method for identifying an agent that decreases a phenotype of a cell including the steps of contacting a cell expressing an MscCa channel with a candidate agent to yield a treated cell and comparing the phenotype of the treated cell with the phenotype of a control cell not contacted with the candidate agent, in which the phenotype is selected from the group of motility, invasiveness, proliferation, or a combination thereof, and a decreased phenotype for the treated cell indicates the candidate agent decreases the phenotype.

The invention also includes a method for treating cancer that includes administering to a subject having cancer an effective amount of a composition including an agent that decreases activity of a mechanosensitive ion channel present on a cancer cell, in which a symptom of the cancer is decreased. The present invention also includes a method for decreasing metastasis of a cancer cell including administering to a subject at risk of developing cancer an effective amount of a composition including an agent that decreases activity of a mechanosensitive ion channel. The invention also includes a method for decreasing a symptom associated with cancer including administering to a subject having cancer an effective amount of a composition including an agent that decreases activity of a mechanosensitive ion channel.

The mechanosensitive ion channel of cancer cells in the methods may be a mechanosensitive Ca²⁺-permeable (MscCa) channel. In an additional embodiment of the methods, the agent may be a polypeptide including an amino acid sequence including at least 90% identity to SEQ ID NO: I or to SEQ ID NO:7. More particularly, the agent may be a polypeptide including SEQ ID NO: 1 or SEQ ID NO:7. The agent used in the methods may also be an antibody that specifically binds an MscCa polypeptide. In an embodiment of this aspect of the invention, the antibody may be an antibody that binds to an epitope present on SEQ ID NO:5 or SEQ ID NO:6. In another embodiment of the cancer treatment methods in which the channel is an MscCa channel, the MscCa channel is an MscCa polypeptide, and the agent is a polynucleotide that decreases expression of the MscCa polypeptide. The cancer treated in particular embodiments of the method may be prostate cancer, breast cancer, colon cancer, lung cancer, bladder cancer, ovary cancer, pancreas cancer, or skin cancer. In an embodiment of the methods in which the channel is an MscCa channel, the agent that decreases the activity of the MscCa channel may be a polypeptide including SEQ ID NO: 2.

The invention also includes a method for inhibiting expression of an MscCa polypeptide that involves administering into a cell an effective amount of an RNA polynucleotide, wherein the polynucleotide includes a sense strand and an antisense strand, wherein the sense strand includes a nucleotide sequence of between 16 and 30 nucleotides, wherein the nucleotide sequence is substantially identical to consecutive nucleotides of an mRNA encoding a polypeptide of SEQ ID NO:2, and wherein the cell including the RNA polynucleotide has decreased MscCa activity, decreased motility, decreased invasiveness, or a combination thereof, when compared to a control cell that does not include the RNA polynucleotide.

The invention also includes a method for treating cancer that involves administering to a subject having cancer an effective amount of an RNA polynucleotide, wherein the polynucleotide includes a sense strand and an antisense strand, wherein the sense strand includes a nucleotide sequence of between 16 and 30 nucleotides, wherein the nucleotide sequence is substantially identical to consecutive nucleotides of an mRNA encoding a polypeptide of SEQ ID NO:2, and wherein a symptom of the cancer is decreased.

The invention also includes a method for decreasing metastasis of a cancer cell that involves administering to a subject at risk of developing cancer an effective amount of an RNA polynucleotide, wherein the polynucleotide includes a sense strand and an antisense strand, wherein the sense strand includes a nucleotide sequence of between 16 and 30 nucleotides, and wherein the nucleotide sequence is substantially identical to consecutive nucleotides of an mRNA encoding a polypeptide of SEQ ID NO:2. The invention also includes a method for decreasing a symptom associated with cancer that involves administering to a subject having cancer an effective amount of an RNA polynucleotide, wherein the polynucleotide includes a sense strand and an antisense strand, wherein the sense strand includes a nucleotide sequence of between 16 and 30 nucleotides, wherein the nucleotide sequence is substantially identical to consecutive nucleotides of an mRNA encoding a polypeptide of SEQ ID NO:2.

Unless otherwise specified, “a,” “an,” “the,” and “at least one” are used interchangeably and mean one or more than one.

### BRIEF DESCRIPTION OF THE FIGURES

FIG. 1; A-D show that mechanosensitive ion channel activity is preserved after frog oocyte membrane protein detergent solubilization and reconstitution in liposomes. A: Channel events recorded from a membrane patch isojated from proteoliposomes composed of solubilized membrane proteins and azolectin phospholipids at a ratio of 1:100. The top trace shows the pressure recording during syringe applied-suction of approximately 30 mmHg. Scale 50 mmHg. The middle trace shows the corresponding membrane patch current, with several current events of 2 pA. The bottom trace shows the continued current trace, in which several unitary current events opened spontaneously (i.e., in the absence of applied suction). The pipette solution was 200 mM KCI, 5 mM Hepes and the bath solution (i.e., facing the inside-out membrane) was 200 mM KCI, 40 mM MgCI₂ and 5 mM Hepes. Pipette potential -80 mV. Scale 2 pA and I second. B: Current-voltage relationship of reconstituted stretch-activated channels with high (40) Mg²+(hollow circles 0, pipette solution, 200 mM K+and 40 mM Mg ²+) and in low (0) external Mg²+(-IDS, pipette solution, 200 mM K+0 mM Mg²+; AVE-O, 200 mM Cs+0 MM Mg²+). In all cases the bath solution had high Mg²+(200 mM K+and 40 MM Mg²+). The solid curves represent fits to inside-out oocyte patch data based on 4-5 patches (data not shown to increase figure clarity) measured under the same ionic conditions (i.e., pipette solution, 200 mM K+, 0 mM Mg²+or 40 mM Mg²+; bath solution, 200 rnM K+, 40 mM Mg²+). C: FPLC profile measured for OG-solubilized oocyte membrane proteins using a UNO Q-l anion-exchange column. D: The percentage of patches showing MscCa activity measured in proteoliposomes with different protein:lipid ratios for proteins from the different FPLC fractions (5 patches tested for each P:L ratio).

FIG. 2 shows stretch-activated multi-channel and single channel currents measured in cell- attached patches from control and hTRPC I -expressing oocytes at different patch potentials. The figure illustrates the current-voltage relation of macroscopic currents from patches from control (3 patches) and hTRPC I injected oocytes (4 patches).

FIG. 3; A-C shows MscCa activity in control and hTRPCI MiRNA-injected oocytes and the effects of hTRPC antisense RNA on native MscCa activity. A: Staircase increase in suction (top trace) applied to a cell-attached patch on a control (water-injected) oocyte activates an inward current (lower trace) of -12 pA. B: Similar to A except cell-attached patch formed on an oocyte injected 4 days previously with hTRPCI transcripts showing activation of -175 pA current. C: Histogram of MscCa/patch density measured in control (water-injected), hTRPCJ cRNA injected, hTRPCI antisense cRNA-injected oocytes and scrambled antisense oligonucleotide injected oocytes (n-30, 15, 20 and IO). Data plotted as mean SEM (p <0.001).

FIG. 4; A-D show that transfection of CHO-KI cells with hTRPCI results in increased MscCa activity. A: Comparison of patches from a hTRPCI-tranfected and a non-transfected cell in response to increasing steps of suction. B: Histogram showing percentage of patches with MscCa activity and MscCa patch density in nontransfected (15) and transfected (21) CHO-KI cells. C: Single channel currents recorded from control and hTRPCI-transfected CHO-KI cells (patch potential -50 mV) indicate the native and expressed channel currents are similar in amplitude. D: Single channel current-voltage of native CHO-KI (filled symbols) and expressed channels (hollow symbols) (0 Mg²+: pipette solution 100 mM KCI, 5 Hepes (KOH) 2 EGTA; I Mg²+: 100 mM KCI, 5 Hepes (KOH), IMgCI₂) FIG. 5; A-C show the conductance and kinetic properties of the MscCa in human PC3 cells. In all patch recordings the upper traces represents the pressure waveform and the lower traces that patch current responses. A: Suction step applied to a cell-attached patch held at -50 mV and 50 mV activates single channel currents. B: Single channel current-voltage relationship of the MscCa measured on cell-attached patches of PC3 cells (solid symbols) and Xenopus oocytes (hollow symbols) with zero Caz+(circles, pipette solution in mM 100 KCI 5 Hepes 2 EGTA) and 1 mnM Ca²+(triangles, pipette solution 100 NaCO 5 Hepes I CaCI₂). C: The MscCa in PC3 cells displays two distinct kinetic modes; a sustained mode in which the channel remains open for the stimulus duration (left hand panel) and a transient mode in which the channels close rapidly (<0.5 s) during pressure step.

FIG. 6; A-E shows the pharmacology of the MscCa in PC3 cells. A: Cell-attached patch recording showing increasing pressure steps (top trace) and the patch current response beginning soon after forming the tight seal (-IO s) and 3 minutes later. The pipette tip was filled (-300 )m form orifice) with Gd³+-free solution and backfilled with 5 tM Gd³+containing solution. B: Histogram showing the % of patches with MscCa activity in the presence of anti-MscCa agents in the pipette solution. C: GsmTx-4 blocks the MscCa; similar protocol to B showing patch currents before and after block by GsmTx-4; in this case MscCa displayed SM gating. D: GsmTx-4 block of MscCa displaying TM gating. E: Patch clamp recording showing MscCa block caused by inclusion of the anti-TRPCI Ab (20 AgmJ) in the solution used to backfill the pipette.
FIG. 7 shows PC3 cell movement and the effects of anti-MscCa agents. Upper panels show representative trajectories before, during, and after application of 5 tM Gd³+, 3 JM GsMTx-4 and 20 gg/ml anti-TRPCI antibody. Lower panels show histograms from 25 or more cells.
FIG. 8 shows the amino acid sequence for the human TRPCI protein; Genbank Accession Number CAA61447 (SEQ ID NO:2).
FIG. 9 shows the nucleotide sequence for the mRNA for human TRPCI protein, Genbank Accession Number X89066 (SEQ ID NO:3).

### DETAILED DESCRIPTION OF PREFERRED EMBODIMENTS OF THE INVENTION

The present invention includes methods for the identification of agents that inhibit the activity of mechanosensitive ion channels. Several aspects of the present invention include the use mechanosensitive ion channels. As used herein, the term “mechanosensitive ion channels” refers to a type of ion channel that is responsive to mechanical stress upon the cell membrane in which the mechanosensitive ion channel is located. Mechanosensitive ion channels may open in response to increased membrane tension, in which case they are categorized as stretch-activated channels, or they may be tonically active and close in response to increased membrane tension, in which case they are categorized as stretch-inactivated cation channels.
Mechanosensitive ion channels (MSCs) have been demonstrated to play an important role in cell motility, and thus present an attractive target for inhibiting the metastasis of tumor cells. Ion channels have also been implicated in regulating other steps in cancer progression, including cell proliferation and apoptosis. The Examples below demonstrate that a mechanosensitive ion channel is present in tumor cells and that it plays a role in the metastatic activity, e.g., the motility and invasiveness, of such cells. Further, the Examples below show that the mechanosensitive ion channel can be inhibited using a variety of agents and methods. Metastasis that is associated with the function of mechanosensitive ion channels can be treated by inhibiting the activity of mechanosensitive ion channels in accordance with the invention. Selective inhibition of mechanosensitive ion channels specifically expressed or overexpressed in tumor cells allows inhibition of tumor cell motility while avoiding the disruption of physiologically important functions such as immunological surveillance and wound healing that could be associated with general inhibition of cell motility.
Mechanosensitive ion channels are a subtype of ion channels. Ion channels can be either anion channels or cation channels. Ion channels include, for example, extracellular ligand-gated, intracellular ligand-gated, voltage gated, inward rectifier, gap junction, and ATP-gated channels. Anion channels are proteins that facilitate the transport of anions across cell membranes. The anions being transported include, for example, chloride, bicarbonate, and organic ions such as bile acids. Cation channels, on the other hand, are proteins that facilitate the transport of cations across cell membranes. The cations being transported may be divalent cations such as Ca+² or Ba+² or monovalent cations such as Na+or K+. By facilitating transport and/or diffusion, ion channels enable particular anions or cations to cross the cell membrane at a greater rate than would normally occur based on simple diffusion through the membrane. While not intending to be bound by theory, it is believed that ion channels contain a receptor site within their pore structure that is specific for the anion(s) or cation(s) that they transport, and that binding of an ion or ions to the receptor site results in a conformation change that allows the bound ion to pass through the membrane, resulting in either passage either into or out of the cell. Ion channels are also referred to as ion transporters.
The mechanosensitive Ca²+-permeable (MscCa) channel is a member of the broad family of Ca²+-permeable channels that may display either strong or relatively weak Ca²+selectivity, and includes voltage-, receptor- and mechano-gated channels as well as the store-operated calcium channels (SOCC). Prior to the present invention, the identity of the protein forming the MscCa was not clear. Members of the transient receptor potential channel (TRPC) family, which includes Drosophila channels (1TRP and TRP-like) and several vertebrate homologs (TRPC 1-7), have been proposed to form SOCC (Montell, C., Mol. Pharmacol., 52, 755-763 (1997)). However, although some TRPCs are sensitive to Ca²+-store depletion and may contribute to store refilling, TRPCs typically display a relatively large single channel conductance (30-80 pS (picosiemens) and are only weakly Ca2+selective, unlike the low conductance (pS) and high Ca2+selectivity first described for SOCC in blood cells (Rarteneck et al., Trends Neurosci. 23, 159-166 (2000)), but similar to MscCa (Hamill & McBride, Pharmacol. Rev., 48, 231-252 (1996)). Furthermore, overexpression of hTRPCI in Xenopus oocytes and in rat liver cells does not increase the endogenous SOCC current, but does increase a Ca²+cation-conductance that is activated by the marine poison maitotoxin (MTX) (Brercton, et al., Biochim. Biophys. Acta., 1540, 107-126 (2001); Brereton, et al., Mol. Cell. Biochem., 214, 63-74 (2000)). Significantly, the MTX-activated conductance shares basic properties with the oocyte MscCa, including cation selectivity, divalent cation block and pharmacology (Bielfeld-Ackermann et al., Pfluegers. Arch. 436, 329-337 (1998)). As described herein, detergent solubilization and functional reconstitution of oocyte membrane proteins shows that an -80 kDa membrane protein identified as TRPC1 is concentrated in the most active protein fraction that reconstitutes MscCa activity. This indicates that TRPCI is MscCa and provides an opportunity to investigate the effects of altering MscCa activity or levels on tumor cell migration.

A preferred mechanosensitive ion channel for use in the present invention is a mechanosensitive Ca²+- permeable (MscCa) channel. Such a channel includes an MscCa polypeptide. As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multipliers (e.g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring. An MscCa 2+polypeptide is part of a channel that mediates the movement of Ca ions into a cell. A mechanosensitive ion channel that includes an MscCa polypeptide may be a homotetramer or may combine with other transient receptor potential channel polypeptides to form a heterotetramer. An example of an MscCa polypeptide is the transient receptor potential channel I (TRPC I) polypeptide, and an example of a TRPC I polypeptide is the amino acid sequence depicted at Genbank accession number CAA61447 (FIG. 8, SEQ ID NO: 2).
The present invention further includes MscCa polypeptides having similarity with the amino acid sequence of SEQ ID NO:2. The similarity is referred to as structural similarity and is generally determined by aligning the residues of the two amino acid sequences (i.e., a candidate amino acid sequence and the amino acid sequence of SEQ ID NO:2) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence is the amino acid sequence being compared to an amino acid sequence present in SEQ ID NO:2. A candidate amino acid sequence can be isolated from an animal, preferably a human, or can be produced using recombinant techniques, or chemically or enzymatically synthesized. Preferably, two amino acid sequences are compared using the Blastp program of the BLAST 2 search algorithm, as described by Tatusova, et a). (FEMS Microbiol. Lett, 174:247-250 (1999)), and available at http://www.ncbi.nlm.nih.gov/blast/b 2seq/b 2.html.
Preferably, the default values for all BLAST 2 search parameters are used, including matrix =BLOSUM62; open gap penalty =I 1, extension gap penalty , gap x-dropoff =50, expect =10, wordsize =3, and optionally, filter on. In the comparison of two amino acid sequences using the BLAST search algorithm, structural similarity is referred to as “identities.” An MscCa polypeptide may include an amino acid sequence having a structural similarity with SEQ ID NO:2 of at least 90 %, for example at least 91%, at least 92%, at least 93%, at least 94%, at least 95%, at least 96%, at least 97%, at least 98%, at least 99%, or at least 100 % identity. An MscCa polypeptide having structural identity to SEQ ID NO:2 typically has the structural features common to the TRP family (Wes et al., Proc. NatI. Acad. Sci. USA, 92:9652-9656 (1995), Minke & Cook, Physiol.
Rev., 82:429-472 (2002)). The structural features include six transmembrane domains, a putative pore region between transnmembrane regions 5 and 6, and the absence of charged residues at every third or fourth position in the fourth transmembrane segment. The characteristics of a transmembrane domain are known and include, for instance, several (e.g., -20) consecutive hydrophobic amino acid residues that allows it to span the thickness of the hydrophobic bilayer. An MscCa polypeptide having structural identity to SEQ ID NO:2 also typically has three ankyrin repeats on the N-terminus (Minke & Cook Physiological Reviews, 82, 429-472 (2002)), and an internal C-terminus in which the 90 terminal amino acids show significant identity to dystrophin, including a coiled-coil structure.

An MscCa polypeptide, such as an MscCa polypeptide having structural identity to SEQ ID NO:2, has MscCa activity. MscCa activity includes the ability to transduce membrane stretch into Ca²+influx. Typically, activation by pressure or suction (at least 20 mrnHg), using, for instance, a patch pipette (Taglietti, V. & Toselli, M.J., Physiol. 407, 311-328 (1988); Yang, X.C. & Sachs, F., J. Physiol. 431, 103-122 (1990)), results in an inwardly rectifying single channel conductance of 50 pS when measured at - 50 millivolts (mV), and 20 pS when measured at +50 mV. MscCa activity typically shows perneant block by Ca²+. Methods for evaluating these characteristics of an MscCa polypeptide include techniques such as patch-clamp or pressure-clamp analysis, which are routine and known to those skilled in the art. MscCa activity also includes the ability to mediate the movement of a cell, the invasiveness of a cell, or the combination thereof. A decrease in activity can result in decreasing motility, invasiveness, or the combination thereof. Thus, MscCa activity can be decreased by preventing a channel from permitting the influx of Ca²+, or decreasing the expression of the channel. MscCa activity can be blocked by 6d³+, by GsmTx-4, a known blocker of MscCa polypeptide activity (Suchyna et al., J. Gen. Physiol., 115:583 (2001)), antibody directed toTRPC1, or the combination thereof.

Identification of Agents

One aspect of the invention includes methods for identifying agents that inhibit the activity of a mechanosensitive ion channel, preferably an MscCa channel. Potential agents suitable for testing are referred to herein as “candidate agents.” The method involves contacting a lipid membrane containing an MscCa channel with a candidate agent and observing whether the activity of the MscCa channel is decreased. The lipid membrane can be, for instance, a lipid membrane present in a cell or in a reconstituted liposome. Methods for the production of reconstituted liposomes are known and routine to the skilled person. A cell that can be used to evaluate a candidate agent may be a cell that expresses an MscCa polypeptide. A cell can be ex vivo or in vivo. As used herein, the term “ex vivo” refers to a cell that has been removed from the body of a subject. Ex vivo cells include, for instance, primary cells (e.g., cells that have recently been removed from a subject and are capable of limited growth in tissue culture medium), and cultured cells (e.g., cells that are capable of extended culture in tissue culture medium). As used herein, the term “in vivo” refers to a cell that is within the body of a subject. Whether a cell expresses a mechanosensitive ion channel can be determined using methods that are routine and known in the art including, for instance, Western immunoblot, ELISA, immunoprecipitation, or immunohistochemistry. Examples of ex vivo cells expressing an MscCa channel include cultured cells such as PC3 (ATCC number CRL-1435) and LnCaP (ATCC number CRL-1740) and primary cells such as prostate carcinoma cells. Other cells can also be modified to express one of the mechanosensitive ion channel polypeptides by introducing into a cell a vector having a polynucleotide encoding the polypeptide.
Inhibition of the activity of mechanosensitive ion channels, preferably, an MscCa channel, can result in several effects, including decreased movement of Ca²+through the channel, decreased motility, decreased invasiveness, decreased cell proliferation, increased apoptosis, or a combination thereof. Methods for determining whether the activity of a mechanosensitive ion channel, preferably, an MscCa channel, is decreased include methods for measuring the movement of Ca2+through the channel. Such methods include techniques such as patch-clamp or pressure-clamp analysis, which are routine and known to those skilled in the art. In another aspect, methods for determining whether activity has decreased include measuring a phenotype of a cell, such as a cell's motility, invasiveness, or the combination thereof. “Motility,” as used herein, refers to the ability of a cell to move across a surface or through a solution under its own direction, as opposed to movement caused by pre-existing currents or other factors such as gravity that might cause the cell to move in response to an external force. “Invasion,” as used herein, refers to the ability of a cell to move through a barrier under its own direction, as opposed to movement due to an external force. Methods for measuring motility of a cell are know and routine and may include visualization methods such as time-lapse videomicroscopy. Methods for measuring invasion are know and routine and may include the use of a Boyden chamber, and/or the use of surfaces coated with barriers. Examples of barriers that can be used include one or more components of basement membrane matrix, for instance the basement membrane preparation commercially available under the tradename MATR1GFL (BD Bioscience). In another aspect, methods for determining whether activity has decreased include measuring changes in cell proliferation or changes in apoptosis. Methods for measuring changes in cell proliferation or changes in apoptosis are known in the art. When a candidate agent is identified that decreases the motility, decreases invasion of a cell, decreased cell proliferation, increases apoptosis, or a combination thereof, the effect of the agent on the activity of an MscCa polypeptide in the cell can then be tested using a method for measuring the movement of Ca²+through the channel, or a method for measuring whether the cell expresses an MscCa channel. Preferably, a candidate agent is able to decrease the activity of a mechanosensitive ion channel, preferably an MscCa channel, by at least 50%, at least 60%, at least 70%, at least 80%, or at least 90%.
Candidate agents may also be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers, including prostate cancer (see, for instance, Polin et al., investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, fbr instance, cancer metastasis, cancer cell motility, cancer cell invasiveness, and the combination thereof.
The sources for candidate agents include, for instance, chemical compound libraries, and extracts of plants and other vegetations. A candidate agent can be, for example, a polypeptide, a polyketide, a polynucleotide, an ion, an organic molecule, an inorganic molecule, or a combination thereof. An example of a polypeptide is antibody. Preferably, the antibody specifically binds an MscCa polypeptide. As used herein, an antibody that can specifically bind a polypeptide is an antibody that interacts only with the epitope of the antigen that induced the synthesis of the antibody, or interacts with a structurally related epitope. An antibody that specifically binds to an epitope will, under the appropriate conditions, interact with the epitope even in the presence of a diversity of potential binding targets. Antibody, either polyclonal or monoclonal, may be prepared using an MscCa polypeptide, preferably, the amino acid sequence depicted at SEQ ID NO:2, or a portion thereof. Preferred portions of an MscCa polypeptide that can be used include residues between the fifth and sixth transmembrane domains, for instance CVGIFCEQQSND (SEQ ID NO:5), and QLYDKGYTSKEQKDC (SEQ ID NO:6).
An example of a polynucleotide is a small inhibiting RNA (siRNA). As used herein, the term “polynucleotide” refers to a polymeric form of nucleotides of any length, either ribonucleotides, deoxynucleotides, or a combination thereof, and includes both single-stranded molecules and double-stranded duplexes. A polynucleotide can be obtained directly from a natural source, or can be prepared with the aid of recombinant, enzymatic, or chemical techniques. Without intending to be limiting, agents can function as substrate mimics (e.g., non-hydrolyzable or substrate trapping inhibitors), antagonists that resemble ions transported by the mechanosensitive ion channel or that interfere with the binding of mechanosensitive ion to substrate ions, or act to prevent ion channel conformation changes induced in response to mechanical stress or substrate ion binding.
A candidate agent that is a polynucleotide can be designed using methods that are routine and known in the art. . For example, polynucleotides that inhibit the expression of a mechanosensitive ion channel, preferably, an MscCa polypeptide, more preferably, the polypeptide depicted at SEQ ID NO:2, may be identified by scanning the mRNA encoding the polypeptide for AA dinucleotide sequences; each AA and the downstream (3) consecutive 16 to 30 nucleotides of the mRNA can be used as the sense strand of a polynucleotide for use as a candidate agent. An example of an mRNA is an mRNA encoding an MscCa polypeptide and depicted at SEQ ID NO:3 The polynucleotide for use as a candidate agent can be substantially identical, preferably, identical, to nucleotides located in the region encoding the polypeptide, or located in the 5or 3untranslated regions of the mRNA. Optionally and preferably, a polynucleotide for use as a candidate agent is modified to include 1, 2, or 3, preferably 1, non-complementary nucleotides. Other methods are known in the art and used routinely for designing and selecting polynucleotides for use as a candidate agent. A polynucleotide for use as a candidate agent may, but need not, begin with the dinucleotide AA at the 5end of the sense strand. A polynucleotide for use as a candidate agent may also include overhangs of 1, 2, or 3 nucleotides, typically on the 3end of the sense strand, the anti-sense strand, or both. Polynucleotides for use as a candidate agent are typically screened using publicly available algorithms (e.g., BLAST) to compare the candidate polynucleotide sequences with coding sequences. Those that are likely to form a duplex with an mRNA other than the mRNA encoding the mechanosensitive ion channel are typically eliminated from further consideration. The remaining polynucleotides may then be tested to determine if they decrease activity of an MscCa polypeptide.
In general, polynucleotides are individually tested by introducing a polynucleotide into a cell that expresses a mechanosensitive ion channel. The polynucleotides may be prepared in vitro and then introduced into a cell.

Methods for in vitro synthesis include, for instance, chemical synthesis with a conventional DNA/RNA synthesizer. Commercial suppliers of synthetic polynucleotides and reagents for such synthesis are well known. Methods for in vitro synthesis also include, for instance, in vitro transcription using a circular or linear vector in a cell free system.

Polynucleotides for use as a candidate agent may also be prepared by introducing into a cell a construct that encodes the polynucleotide. Such constructs are known in the art and include, for example, a vector encoding and expressing a sense strand and an anti-sense strand of a polynucleotide, and RNA expression cassettes that include the sequence encoding the sense strand and an anti-sense strand of a polynucleotide flanked by operably linked regulatory sequences, such as an RNA polymerase III promoter and an RNA polymerase III terminator, that result in the production of an RNA polynucleotide. Methods for introducing a polynucleotide, including a vector or RNA expression cassette encoding a polynucleotide, are known in the art and routine.

A polynucleotide may include a spacer, made up of nucleotides, located between the sense and anti-sense strand. Such a polynucleotide is often referred to in the art as a short hairpin RNA (shRNA). Upon base pairing of the sense and anti-sense strands, the spacer region forms a loop. The number of nucleotides making up the loop can vary, and loops between 3 and 23 nucleotides have been reported (Sui et al., Proc. Natl. Acad. Sci. USA, 99, 5515-5520 (2002), and Jacque et al., Nature, 418, 435-438 (2002)).
A polynucleotide used in the methods of the invention can be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, A Molecular Cloning: A Laboratory Manual, Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, or artificial chromosome vectors. Examples of viral vectors include, for instance, adenoviral vectors, adeno-associated viral vectors, lentiviral vectors, retroviral vectors, and herpes virus vectors. A vector may result in integration into a cell's genomic DNA. Typically, a vector is capable of replication in a bacterial host, for instance E. coli. Preferably the vector is a plasmid. A polynucleotide of the present invention can be present in a vector as two separate complementary polynucleotides, each of which can be expressed to yield a sense and an antisense strand of the polynucleotide, or as a single polynucleotide containing a sense strand, a loop region, and an anti-sense strand, which can be expressed to yield an RNA polynucleotide having a sense and an antisense strand.
Selection of a vector depends upon a variety of desired characteristics in the resulting construct, such as a selection marker, vector replication rate, and the like. Suitable host cells for cloning or expressing the vectors herein are prokaryote or eukaryotic cells. Preferably the host cell secretes minimal amounts of proteolytic enzymes. Suitable prokaryotes include eubacteria, such as gram-negative organisms, for example, E. coli.
An expression vector optionally includes regulatory sequences operably linked to the polynucleotide of the present invention. Typically, the promoter results in the production of an RNA polynucleotide. Examples of such promoters include those that cause binding of an RNA polymerase III complex to initiate transcription of an operably linked polynucleotide of the present invention. Examples of such promoters include U6 and H I promoters. Vectors may also include inducible or regulatable promoters for expression of a polynucleotide of the present invention in a particular tissue or intracellular environment. The polynucleotide of the present invention also typically includes a transcription terminator. Suitable transcription terminators are known in the art and include, for instance, a stretch of 5 consecutive thymidine nucleotides.
When evaluating whether a polynucleotide functions to inhibit expression of a mechanosensitive ion channel, the amount of target mRNA (i.e., the specific mRNA whose expression is to be inhibited by the polynucleotide) in a cell containing a polynucleotide can be measured and compared to the same type of cell that does not contain the polynucleotide. Methods for measuring mRNA levels in a cell are known in the art and routine. Such methods include quantitative reverse-transcriptase polymerase chain reaction (RT-PCR).

Primers and specific conditions for amplification of an mRNA vary depending pon the mRNA, and can be readily determined by the skilled person. Other methods include, for instance, Northern blotting, and array analysis.
Other methods for evaluating whether a polynucleotide functions to inhibit expression of mechanosensitive ion channels includes monitoring for the presence of the channels. For instance, assays can be used to measure a decrease in the amount of the mechanosensitive ion channel. Methods for measuring a decrease in the amount of a mechanosensitive ion channel include Western immunoblot, immunoprecipitation, or immunohistochemistry.

Treatment of Cancer by Inhibition of Activity or Expression of Mechanosensitive Ion Channels The observation that mechanosensitive ion channels, for instance, MscCa channels, described herein play a role in motility and invasion of cells indicate these channels may be a suitable target for methods of treating cancer. Cancer, as referred to herein, includes the diseases or disorders characterized by the presence of abnormal cells dividing without the control present in healthy cells.
The threat of cancer is greatly magnified by metastasis of cancer cells.
Cancer that remains at its original site, even if cell proliferation is fairly rapid, can be treated by removal or in situ treatment of the cancerous tissue. Metastasis not only multiplies the number of sites at which treatment must occur, but also makes it unclear after treatment whether all cancer cells have been removed, as tumors often take some time to manifest themselves. Should metastasis occur at a sufficiently high level, surgical treatment rapidly become impossible and treatment by other methods generally becomes much more difficult. The existence of metastasis has led to the creation of additional cancer nomenclature. A tumor formed by cells that have spread is referred to herein as a “metastatic tumor,” whereas the initial and original source of the tumor cells is referred to herein as a primary tumor. Accordingly, a primary tumor can include cancerous cells that are not metastatic, and cancerous cells that are metastatic. As used herein, a metastatic cell is a cancer cell that exhibits motility and migratory capacity sufficient to carry out the steps involved in metastasis; namely, invasion, detachment, embolization, and extravasation, and is hence capable of forming a metastatic tumor. A primary tumor that includes metastatic cells is also referred to as a malignant tumor. A benign tumor is a primary tumor that does not include metastatic cells. Typically, only metastatic cancerous cells migrate from the primary tumor and establish metastatic tumors. Metastatic tumors can also shed metastatic cells and thus function as the source for further metastatic tumor cells.
Tumor metastasis involves various types of cell movement. For example, the metastasis of cells from a primary tumor may require invasion of nearby tissue to allow the metastatic cells to penetrate blood vessels and enter circulation. Metastatic cells then move in circulation until they adhere to a different site within the body. Metastatic cells must then move again, penetrating the adjacent tissue (“extravasation”) and leave circulation to form a metastatic tumor site. While the cells of a primary tumor can theoretically metastasize by simply detaching from the primary tumor and relocating themselves via the circulatory or lymphatic systems, it is possible that directed movement of the cell through tissue is required, both at the initial stage before detachment and during extravasation to form a metastatic tumor site, particularly because many cancer cells must form a microenvironment isolated from the circulation in order to survive.
Experiments have demonstrated that MscCa activity is required for cell motility in the tested tumor cells, as shown in the Examples below. As cell motility is involved in the formation of metastic tumors, treatments that result in an inhibition in the activity of mechanosensitive ion channels are expected to result in the prevention or inhibition of metastatic tumors in a cancer patient. The invention thus provides a method for treating cancer in a mammal, preferably a human. The method is also well suited for veterinary applications such as treatment of cancer in a pet such as a cat or a dog. The method is effective to treat a cancer characterized by cells that express mechanosensitive ion channels, particularly metastatic cells from cancers originating in various 5 tissues including, for example, carcinomas, sarcomas, leukemias, and lymphomas. Examples include cancers of the prostate, breast, colon, lung, bladder, ovary, pancreas and skin. Preferably, the treated cancer is prostate cancer. The stages generally used to categorize prostate cancer include localized, in which the tumor is nonpalpable or is palpable but confined to the 10 prostate gland; regional, in which the tumor has grown through the prostate capsule and into, for instance, seminal vesicles or nearby muscles and organs;
and metastatic, which includes tumors that have metastasized to the pelvic lymph nodes, more distant parts of the body, or the combination thereof. The term “prostate cancer,” as used herein, refers to all three of these stages of cancer, as well as precancerous conditions likely to lead to prostate cancer, including enlarged prostate and prostatic intraepithelial neoplasia.
Typically, whether a subject has a cancer, and whether a subject is responding to treatment, is determined by evaluation of symptoms associated with the cancer. As used herein, the term “Symptom” refers to objective evidence of a cancer present in a subject. Symptoms of cancer include, for instance, the presence and size of a primary tumor, the presence of metastatic cells present in a primary tumor, the presence, size, and number of metastatic tumors, and the presence and amount of biomarkers. Biomarkers are compounds, typically polypeptides, present in a subject and indicative of the progression of a cancer. An example of a biomarker for prostate cancer is prostate specific antigen (PSA). The evaluation of symptoms of cancer are routine and known in the art. A subject may have one symptom, or a combination of two or more symptoms. Accordingly, the present invention is also directed to, for instance, methods for decreasing the size of a primary tumor, decreasing the number of metastatic cells in a primary tumor, decreasing metastatic tumor formation, decreasing the motility of a metastatic cell, decreasing invasion of a metastatic cell, or a combination thereof.
Treatment can be prophylactic or, alternatively, can be initiated after the development of cancer. Treatment that is prophylactic, for instance, initiated before a subject manifests cancer symptoms, is referred to herein as treatment of a subject that is “at risk” of developing a disease. An example of a subject that is at risk of developing cancer is a person having a risk factor, such as a genetic marker, that is associated with the disease. Examples of genetic markers indicating a subject has a predisposition to develop certain cancers include alterations in the BRAC I and/or BRAC2 genes (breast, prostate, or colon cancer) and HPCI (prostate cancer). Treatment can be performed before, during, or after the occurrence of cancer. Treatment initiated after the development of cancer may result in decreasing the severity of the symptoms of one of the conditions, or completely removing the symptoms. The methods include administration of a composition including an amount of one or more agents effective to decrease the activity of a mechanosensitive ion channel, preferably, an MscCa channel. The agent can be introduced into the mammal either systemically or at the site of a cancer tumor. As used herein, an “effective amount” is an amount effective to inhibit the activity of mechanosensitive ion channels, either by blocking channels or decreasing their number, decrease a symptom associated with a cancer, or a combination thereof. The agent, if a polynucleotide, may be introduced into a cell as an RNA polynucleotide, or as a vector including a DNA polynucleoticle that encodes and wiji express the RNA polynucleotide. More than one type of agent can be administered. For instance, two or more polynucleotides designed to silence the same mRNA can be combined and used in the methods described herein. Alternatively, two or more agents can be used together that effect different targets, such as using a mechanosensitive ion channel blocking agent alongside a polynucleotide designed to silence an mRNA. Whether an agent is expected to function in the methods of the present invention can be evaluated using ex vivo models and animal models, as described herein above. Such models are known in the art and are generally accepted as representative of disease or methods of treating humans. The agent can be administered with a therapeutic agent, for instance, a chemotherapeutic or radiotherapeutic agent. Suitable chemotherapeutic and radiotherapeutic agents are known to the art. Optionally, the agent can be linked to a chemotherapeutic or radiotherapeutic agent, thereby possessing the dual activities of inhibiting a mechanosensitive ion channel and serving as a carrier molecule for the cytotoxic drug. Where the resulting molecular complex includes a cleavable therapeutic agent, treatment can include delivery of yet another agent to effec( cleavage.

Examples of agents that can be administered in the methods of the present invention include those identified using the methods described herein. Preferred agents include, for example, amiloride and analogs thereof such as dimethylamiloride, bromohexamethyleneamiloride, phenamil, hex amethyJeneamiloride, 6-iodide-2-methoxy-5-nitrobenzamine, and 5-(N-propyl-N-butyl)-dicholl; aminoglycoside antibiotics such as streptomycin, neomycin, and gentamicin, and members of the lanthide series (the elements between lanthanum and lutetium, inclusive). In some aspects of the present invention, a member of the lanthide series does not include gadolinium. Polynucleotides may also provide suitable agents. An example of a polynucleotide-based agent is the polynucleotide obtained from Ambion (Austin, TX) as product number 7216, 731 1, or 7404. Another example of an agent includes an antibody that binds to an epitope present in the amino acid sequence SEQ ID NO:2, preferably CVGEFCEQQSND or QLYDKGYTSKEQKDC. An example of such an antibody is an anti-TRPCI antibody commercially available from Alomone Labs (Jerusalem, Israel). Another example of an agent is the polypeptide GCLEFWWKCNPNDDKCCRPKLKCSKLFKLCNFSSG (SEQ ID NO: I). This polypeptide, referred to as GsMTx-4, is a specific and potent blocker of MscCa channels (see Example 4) and is known to be an antiarrhythmic agent (see Sachs et al., U.S. Patent Application No. 2002/0077286). Another polypeptide is YCQKWMWTCDEERKCCEGLVCRLWCKKKIEW (SEQ ID NO: 7). This pol ypeptide, referred to as GsMTx- 1, is also known to be an antiarrhymic agent (U.S. Pat. No, 5,756,663, issued to Lampe et al.). Other polypeptides that can be used as an agent in the present invention include polypeptides having similarity with the amino acid sequence of SEQ ID NO: I or SEQ ID NO:7, preferably, SEQ ID NO: 1. This similarity is referred to as structural similarity and may be determined as described herein above by aligning the residues of the two amino acid sequences (i.e., a candidate amino acid sequence and the amino acid sequence of SEQ ID NO: 1 or SEQ ID NO:7) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from an animal or insect, or can be produced using recombinant techniques, or chemically or enzymatically synthesized. Such polypeptides may include an amino acid sequence having a structural similarity with SEQ ID NO: I or SEQ ID NO: 7 of at least 90 %, for example at least 91%, at least 92%, at least 93%, at least 94%, at least 95%, at least 96%, at least 97%, at least 98%, at least 99%, or at least 100 % identity. A polypeptide having structural identity to SEQ ID NO: I typically has the structural features common to many other peptide toxins from both terrestrial and aquatic animal venoms (Narasimhan et al., Nature Structural Biol., 1:850-852 (1994), and Norton et al., Toxicon, 36:1573-1583 (1998)). The structural features include six cysteine residues arrayed in an inhibitor cysteine knot (ICK) motif (Ostro et al., Toxicon 42, 263-274 (2003)). An optional structural feature is the conservation of a phenylalanine at position 5, an aspartic acid at position 13, a leucine at position 20, or the combination thereof. A further optional structural feature is an overall positive charge. A polypeptide having structural similarity with SEQ ID NO: I or SEQ ID NO:7 inhibits the activity of a mechanosensitive ion channel, preferably an MscCa channel, more preferably, an MscCa channel including an MscCa polypeptide.
The methods of treatment may be used in conjunction with other methods such as, for instance, radiation therapy, hormonal therapy, surgery, cryosurgical ablation, the use of other agents that target a non-metastatic aspect of tumor cell activity, such as cell proliferation, angiogenesis, or suppression of apoptosis, or a combination thereof. The agents may be administered in any order or may be administered simultaneously (co-administration). Multiple agents that target mechanosensitive ion channels and/or non-metastatic aspects of tumor cell proliferation may be administered. A wide variety of antitumor agents are available that may be used as a second, supplemental agent, to complement the activity of agents described herein that affect mechanosensitive ion channels (see, for example, Fischer et al., eds., The Cancer Chemotherapy Handbook, 6th ed., (2003)). Antitumor agents that have proven particularly effective in treating prostate cancer include, for instance, cyclophosphamide, methotrexate, doxorubicin, 5-fluorouracil, cisplantin, mitomycin C, and decarbazine. Arrest or reversal of cell growth or proliferation by agents can be evidenced by various phenotypic changes in the cancer cells such as increased differentiation, decreased affinity for ECM proteins, increased cell-cell adhesions, slower growth rate, reduced numbers of mechanosensitive ion channels, decreased cell migration or invasion, and can be caused either directly or indirectly.
Preferred methods for administering one or more agents of the present invention include administration during surgery, for instance surgery to resect a cancerous part, organ, system, or combination thereof, of a subject. For treatment of prostate cancer, surgeries include, for example, radical prostatectomy or bilateral orchiectomy. After removal of cancer cells, tissue in the surrounding area can be perfused with a solution containing one or more of the agents of the present invention, or an implant containing one or more of the agents of the present invention can be placed near the area of resection. The agents may also be administered by other methods known in the art. For instance, agents may be administered systemically.
The present invention is also directed to compositions including an agent of the present invention. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.
A composition containing an agent of the present invention may be prepared by methods well known in the art of pharmacy. In general, a composition can be formulated to be compatible with its intended route of administration. Examples of routes of administration include perfusion and parenteral, e.g., intravenous, intraderrnal, subcutaneous, oral (e.g., inhalation), transdermal (topical), transmucosal, and rectal administration. Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.
Compositions can include sterile aqueous solutions (where water soluble) or dispersions and sterile powders for the extemporaneous preparation of sterile solutions or dispersions. For intravenous administration, suitable carriers include physiological saline, bacteriostatic water, Cremophor EL (BASF, Parsippany, N.J.) or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for examnple, aluminum monostearate and gelatin.
Sterile solutions can be prepared by incorporating the active compound (e.g., an agent) in the required amount in an appropriate solvent with one or a combination of ingredients enumerated above, as required, followed by filtered sterilization. Generally, dispersions are prepared by incorporating the active 3 compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.
Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.
For administration by inhalation, the active compounds are delivered in the form of an aerosol spray from a pressured container or dispenser that contains a suitable propellant, e.g., a gas such as carbon dioxide, or a nebulizer.
Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.
The active compounds can also be prepared in the form of suppositories (e.g., with conventional suppository bases such as cocoa butter and other glycerides) or retention enemas for rectal delivery.
Polynucleotide agents can also be administered by any method suitable for administration of polynucleotide agents, e.g., using gene guns, bio injectors, and skin patches as well as needle-free methods such as the micro-particle DNA vaccine technology disclosed by Johnston et al. (U.S. Pat. No. 6,194,389). Additionally, intranasal delivery is possible, as described in, for instance,
Hamajima et al. Cli n. Immunol. Immunopathol., 88, 205-2 0 (1998).
Liposomes and iiiicroencapsulation can also be used.
The active compounds may be prepared with carriers that wiji protect the compound against rapid elimination from the body, such as a controlled release formulation, including implant. Biodegradable, biocompatible polymers can be used, such as ethylene vinyl acetate, polyanhydrides, polyglycolic acid, collagen, polyorthoesters, and polylaclic acid. Such formulations can be prepared using standard techniques. The materials can also be obtained commercially from, for instance, Alza Corporation and Nova Pharmaceuticals, Inc. Liposomal suspensions can also be used as pharmaceutically acceptable carriers. These can be prepared according to methods known to those skilled in the art.
Toxicity and therapeutic efficacy of such active compounds can be determined by standard pharmaceutical procedures in cell cultures or experimental animals, e.g., for determining the LDso (the dose lethal to 50% of the population) and the ED₅(the dose therapeutically effective in 50% of the population). The dose ratio between toxic and therapeutic effects is the therapeutic index and it can be expressed as the ratio LDso/ED. Compounds that exhibit high therapeutic indices are preferred. The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the methods of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.
The compositions containing an agent can be administered one or more times per day to one or more times per week, including once every other day. The skilled artisan will appreciate that certain factors may influence the dosage and tin-ng required to effectively treat a subject, including but not limited to the severity of the disease or disorder, previous treatments, the general health and/or age of the subject, and other diseases present. Moreover, treatment of a subject with an effective amount of an agent can include a single treatment or, preferably, can include a series of treatments.

Kits for Administration of Agents

The present invention also provides a kit for practicing the methods described herein. The kit includes one or more of the agents of the present invention in a suitable packaging material in an amount sufficient for at least one administration. Optionally, other reagents such as buffers and solutions needed to practice the invention are also included. Instructions for use of the packaged agents are also typically included.
As used herein, the phrase “packaging material” refers to one or more physical structures used to house the contents of the kit. The packaging material is constructed by well known methods, preferably to provide a sterile, contaminant-free environment. The packaging material has a label that indicates that the agent(s) can be used for the methods described herein. In addition, the packaging material contains instructions indicating how the materials within the kit are employed to practice the methods. As used herein, the term “package” refers to a solid matrix or material such as glass, plastic, paper, foil, and the like, capable of holding within fixed limits the agent(s). Thus, for example, a package can be a glass vial used to contain appropriate quantities of the agents(s). “Instructions for use” typically include a tangible expression describing the conditions for use of the agent.
The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Identification of TRCP1 as MscCa Protein in Oocytes

Detergent-solubilization of frog oocyte membrane proteins followed by liposome reconstitution and patch-clamp evaluation was used to identify the protein forming MscCa. The first step was the preparation of enriched frog oocyte plasma membranes. Female frogs (Litoria moorei or Xenopus laevis) were anaesthetized and then euthanized by decapitation. After surgical removal, the oocytes were initially stored in Barth's medium (defined in Zhang et al., J. Physiol., 523.1, 83-99 (2000)) overnight at 18° C. Oocyte plasma membranes were isolated according to Wall and Patel (J. Memb. Biol., 107, 189-201 (1989)), with a few minor modifications. Stages I-VI oocytes from 2-3 frogs were homogenized in homogenizing buffer (0.25 M sucrose, 10 mM Hepes, 1 mM ethylene glycol bis(-aminoethyl ether)-N,N,N′N′-tetraacetic acid (EGTA), 2 MM MgC₂ I mM PMSF pH 7.4) and allowed to settle by gravity. A white pellet was then removed and placed in hypotonic buffer (2 mM MgC, 1 μM PMSF, pH 7.4) and homogenized with 20 strokes of a loose-fitting pestle to dissociate cortical granules. Membranes were then washed in high-salt buffer (0.7 M NaCl, 10 mM Hepes, pH 7.4) followed by a low-salt wash (10 mM Tris, I mM EDTA, pH 7.4). Plasma membrane pellets were stored at -80° C. in storage buffer (50 mM KCI, I mM EGTA, 5 mM Hepes, 10% glycerol, pH 7.4). The next step involved detergent solubilization, fractionation and Western blots. To prepare detergent-solubilized oocyte membrane proteins, the procedures of Sukharev et al. were followed (Sukharev et al., Biophys. J., 65, 177-183 (1993)). The plasma membrane samples were thawed, pelleted, and then placed in Bis-Tris buffer containing 1.5% n-Octyl-p-glucoside at 4° C. for 24 hours on a platform rocker. The suspension was then ultracentrifuged at 90,000 RPM in a T-LIOO centrifuge (Beckman, 100.4 rotor) for 30 min. The supernatant was concentrated using a stirred Ultrafiltration Cell (Millipore, Bedford, MA). To fractionate the membrane proteins, FPLC was performed using a UNO Q- I anionexchange column (BIO-RAD, Hercules, CA) on the concentrated sample, and fractions were collected. Peak fractions were further concentrated and run on a 12% S1)S polyacrylamide gel and silver stained.

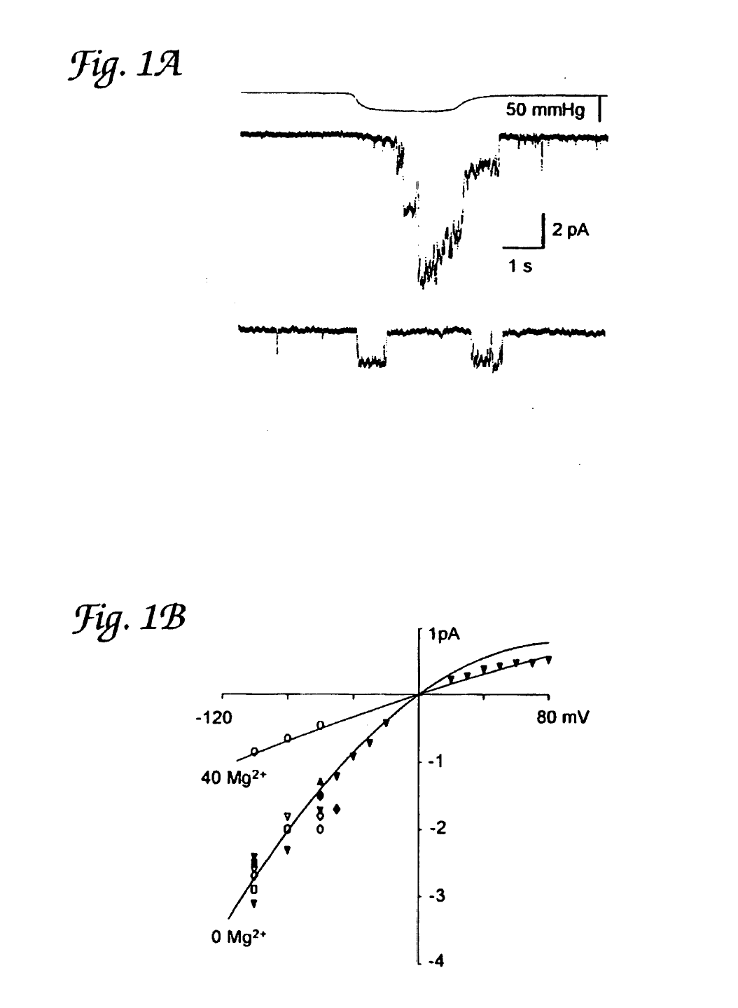

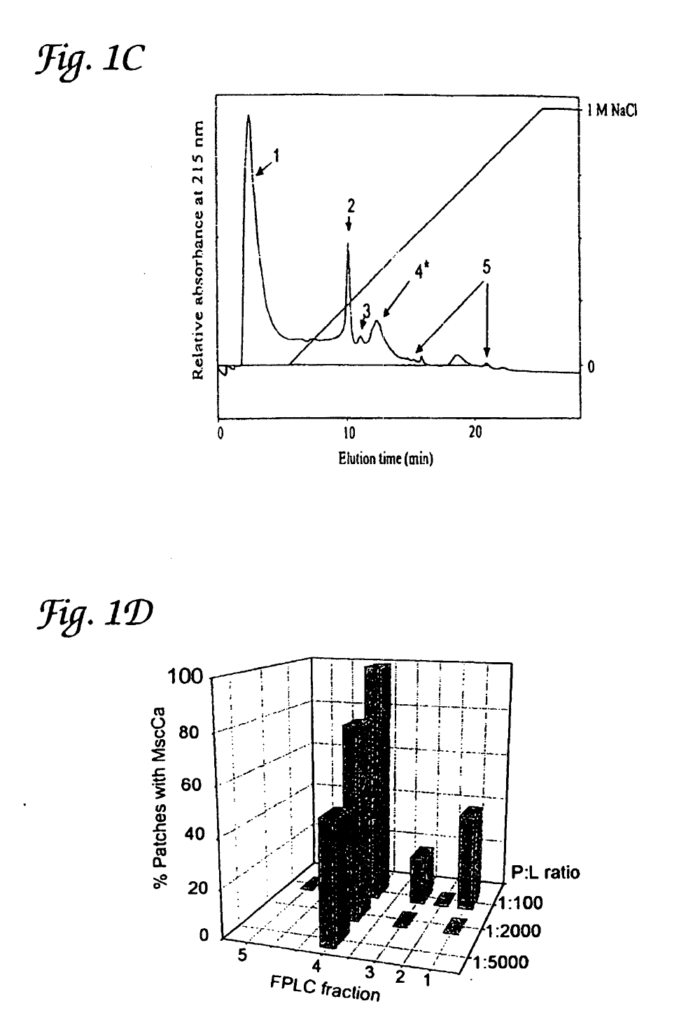

For Western blot experiments on CHO-KI cells, Iysates were collected in ice-cold buffer (10 mM Tris, 150 mM NaC, S5 μM EDTA, I M KI, I mM PMSF, 2.5 mg/ml protease inhibitor cocktail (Sigma # P 8340), and 1.5% n-Octyl-p-glucoside) and vigorously shaken at 4° C. for 15 minutes and then centrifuged at 14,000 RPM, 4C., 30 min. The supernatants were stored at -80° C. until used. The amount of protein was determined using the bicinchoninic acid (BCA) protein assay (Pierce BIO-RAD, Rockford, IL). Two different anti-TRPCI antibodies were used to identify TRPC1 in Westerns. One antibody was raised against the Tle3 epitope (CVGIFCEQQSND) (SEQ. ID NO: 5) that lies between the S5 and S6 membrane spanning domains, the proposed to pore region of the channel as described by Wes et al. (Proc. Nat. Acad. Sci. USA, 92, 9652-9656 (1995)). The other was a commercial anti-TRPCI antibody (Alomone Labs. Lot # AN-02) raised against the peptide QLYDKGYTSKEQKDC (SEQ. ID NO: 6) (amino acid residues 557-571 of hTRPCI, accession # P48995). Both antibodies identified a -80 kDa protein in the FPLC fraction. Proteins were transferred to polyvinylidene fluoride (PVDF) blots and developed by enhanced chemiluminescence. Blol incubation with primary antibodies was carried out overnight in the cold room by gentle shaking. The antigenic peptide was used to specifically block antibody protein binding.
Finally, liposome reconstitution of membrane proteins and patch clamp assay were carried out. Membrane proteins were reconstituted in liposomes for patch clamp recording according to Sukharev et al. (Biophys. J., 65, 177-183 (1993)). Phosphatidylcholine (azolectin) was dissolved in chloroform and small aliquots of lipid were dried under nitrogen, resuspended in 200 mM KCl, 5 mM Hepes, pH 7.2 and bath-sonicated for 5 min. A 200 ii volume (equivalent to 2 mg lipid) was added to the membrane protein to achieve protein to lipid ratios ranging from 1:50 down to 1:5000, and left on a platform rocker for one hour at room temperature. Biobeads (Calbiochem, San Diego, CA) were then added to remove n-octyl-p-glucoside, and the suspension was rocked for a further 3 hours at room temperature. The liposomes were collected by ultracentrifugation at 90,000 RPM in a T-L 100 centrifuge (Beckman, 100.4 rotor) for 30 minutes and resuspended in 200 mM KCI, 5 mM Hepes, pH 7.2. Aliquots of the liposomes were spotted onto glass slides and allowed to dehydrate under vacuum at for 6 hours followed by overnight rehydration (200 mM KCI, 5 mM Hepes, pH 7.2) under humid conditions.
For liposome recording, standard patch-clamp techniques were used. Pipettes were filled with either low MgCI₂ (200 mM KCI or 200 mM CsCl, 5 mM Hepes, pH 7.2 adjusted with KOH) or high MgCI₂ recording solution (200 mM CsC, 40 mM MgCI₂, 5mM Hepes, pH 7.2 adjusted with KOH). A small aliquot (1-2 gl) of rehydrated liposonies was placed in the 0.5 ml patch-clamp chamber containing high MgCl₂ solution. The patch pipette tip was gently touched against unilamellar blisters, which arise spontaneously from the liposomes in the high MgCI₂ solution, and suction was applied. Seals (>20 GO) either formed immediately or after application of a brief pulse of negative pressure (<50 mmHg) applied to the interior of the patch pipette. To record single channel currents, inside-out patches were formed by passing the pipette tip briefly through the solution-air interface. Channel activation was achieved by applying pressure (suction) pulses of -10 to -200 mmHg by mouth or syringe and was measured with the piezoelectric pressure transducer (Omega Engineering, Bridgeport, NJ). Single channel currents were filtered at 2 kHz, digitized at 5 kHz and analyzed using pCLAMP6 data acquisition and analysis software (Axon Instruments, Foster City, CA). Current recordings were viewed with the Axoscope for Windows program (Axon Instruments).
In order to identify the specific protein(s) that may underlies the observed calcium transport (presumably by MscCa) activity, membrane protein fractionation using FPLC was carried out and a protein profile with several distinct peaks was obtained. Fig. IC shows a chromatogram of the observed protein elution pattern selected from chromatograms of three different frog preparations. Liposome reconstitution of the proteins representing several of the main peaks showed MscCa activity when reconstituted with a relatively high protein to lipid ratio (1:100; Fig. ID). However, only one fraction (fraction 4*) retained activity when the ratio was reduced to 1:2000 or 1:5000. A similar peak MscCa active fraction with a conductivity of 16 mS/cm was obtained from the 2 other frog oocyte preparations. A silver-stained gel of the fractionated proteins showed that the most active fraction displayed the highest abundance of a -80 kDa protein. The 80 kDa protein band was found to contain TRPC1 by immunological means.
MscCa measured in cell-attached patches on frog oocytes showed little or no spontaneous openings, but can be experimentally activated by pressure or suction (>20 mmHg) applied to the patch pipette. Once activated, the channel displays a unitary chord conductance of 40 pS (measured at −50 mV) in normal Ringer's, and shows permeant ion block by divalent cations. This activity was preserved after oocyte membrane proteins had been solubilized in the detergent n-octyl-p-glucoside (OG) and reconstituted in liposomes, as described above. Figure IA show patch-clamp current recordings from an “inside-out” isolated liposome patch at -80 mV in response to pressure (-30 mmHg) applied to the pipette. In this case, the membrane proteins were reconstituted at a protein:lipid ratio of 1: 100. The pressure pulse activated at least 3 unitary current events of -2 pA. A low frequency (˜1s⁻¹) of spontaneous current events of similar amplitude was recorded on the same patch (FIG. 1A lower trace). Similar current events activated by pressures of 20-50 mmHg were recorded in 38 other patches formed on proteoliposomes of different composition. In contrast, patches of pure liposomes failed to express unitary current events even with applied pressures as high as 200 mmHg, which ultimately ruptured the patch/seal (5 out of 5 patches tested). The single channel current-voltage relationship measured under symmetrical 200 mM K+with 40 nmM internal Mg²+(high Mg2+was necessary to cause liposome blebbing, a critical requirement for patch clamp recording) indicated an inwardly rectifying channel (-30 pS at -50 mV and -5 pS at 50 mV) that reversed at 0 mV, and was similar to the MscCa recorded from inside-out oocyte patches under the same high internal Mg²+ionic conditions (solid curve, FIG. I B). Replacing external K+with Cs+did not significantly change inward currents, whereas inclusion of 40 MM Mg2, in the external (pipette) solution significantly reduced inward conductance (-10 pS at -50 mV, FIG. 1B). These conductance properties and pressure sensitivity of the reconstituted channel were consistent with the properties of the oocyte MscCa as described by Talietti et al. (J. Physiol., 407, 311-328 (1988)) and others.

### Example 2

### Characterization of TRPC1 present in oocytes

Heterologous expression of the human TRPCI resulted in a >1000% increase in MscCa patch density, whereas injection of a TRPC I-specific antisense RNA abolished endogenous MscCa activity. hTRPCI transfection of CHO-KI cells also significantly increased MscCa expression. The results were obtained as described below.
Heterologous expression of hTRPCI and antisense in the Xenopus oocyte 5 was achieved using the following method. The hTRPCI cDNA, described by Wes et al. (Proc. Nat. Acad. Sci. USA, 92, 9652-9656 (1995)) was incorporated in the dual-function plasmid-vector, pXOOM (Xenopus oocyte or mammalian) described by Jespersen et al. (Biotechniques, 32, 536-540 (2002)), that allows expression of both the sense and antisense version. HTRPCI cRNA and antisense cRNA were made according to standard procedures using T7 and SP6, respectively, nMessage mMachine kits (Ambion, Austin, Tex.), and injected into oocytes three days prior to recording (25 ng RNA/oocyte). Control oocytes were injected either with water or with the scrambled antisense oligonucleotide 5′-CTTWTG ACC GCT CAT CCC TAT AGT ATT TGC-3′ (SEQ ID NO: 4). Transfection of CHO-KI cells was carried out as follows. CHO-KI cells (ATCC number CCL 61) were cultured in RPMI 1640 medium, supplemented with 5% FCS, sodium pyruvate, glucose and antibiotics at 37° C. and 5% CO₂.
Cell transfection with pXOOM-hTRP(CI was carried out using Lipofectamine 2000 (Invitrogen, Carlsbad, CA) according to the manufacture instructions. In 20 brief, 80-90% confluent cells were incubated overnight in the presence of the lipofectamine-plasmid complexes. Serum was added only after the first 5 hours of incubation. A transfection efficiency of 50% was detected by GFP fluorescence. Transfected cells were subsequently subcloned in the presence of 1 mg/ml Geneticin G418 (Invitrogen, Carlsbad, CA) for 12 days. Selected 25 colonies were maintained afterwards in culture medium plus 0.5 mg/ml Geneticin G418.

The pressure-clamp technique was used to activate single MscCa currents in cell-attached membrane patches on Xenopus oocytes and CHO-K 1, using the method described in McBride et al. (Pfluigers Archiv., 421, 606-612 (1992)). Patch pipettes were pulled on a Flaming Brown P-87 puller (Sutter, Novato, CA) to have a tip diameter of -2 μm. The pipette solution contained 100 mM KC1, 2 niM EGTA (KOH) and 5 mM Hepes (KOH). When testing Gd³+the EGTA was left out of the pipette solution. Transfected CHO-KI cells (eGFP positive cells) were selected for patching under a fluorescence microscope.

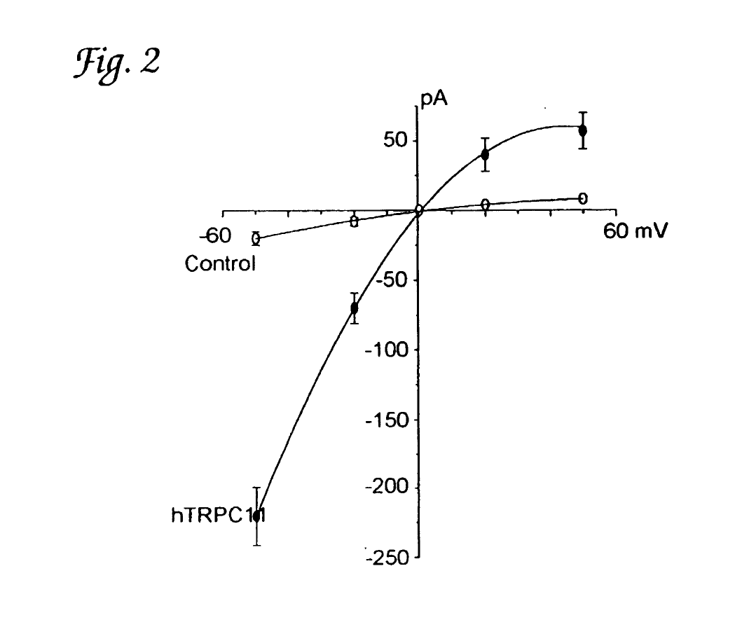

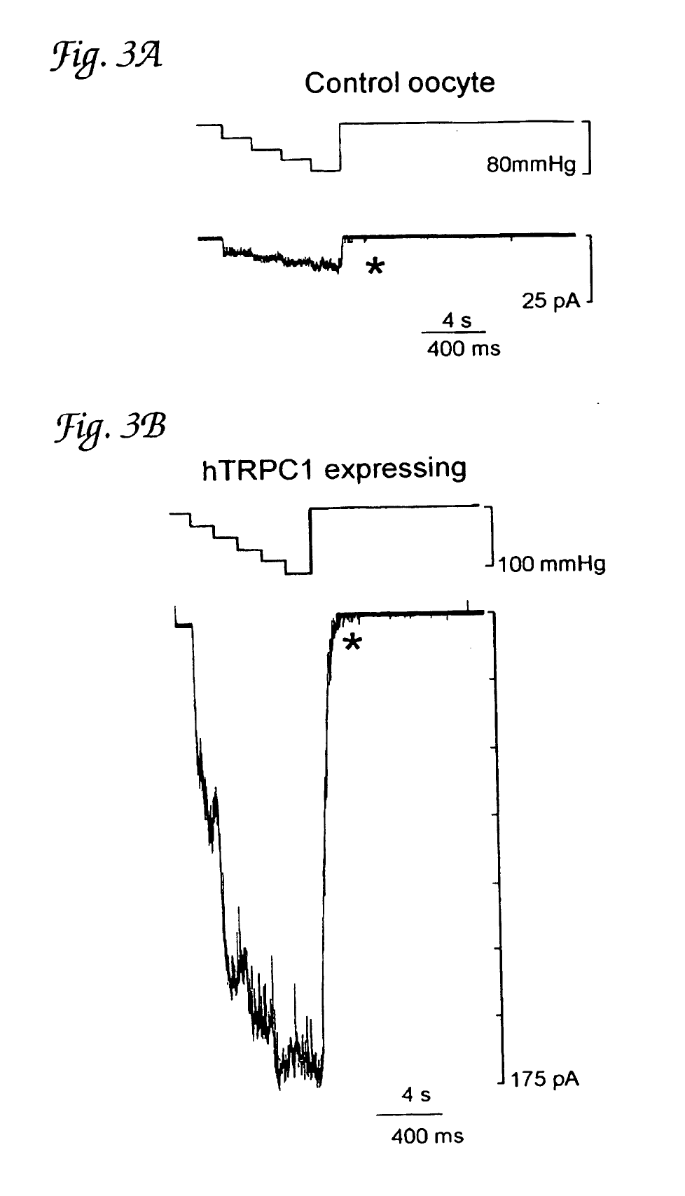

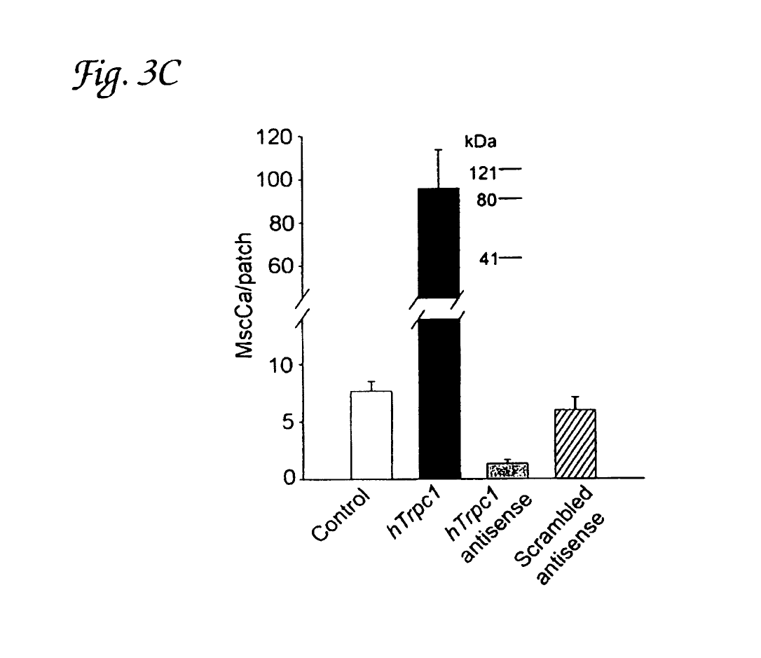

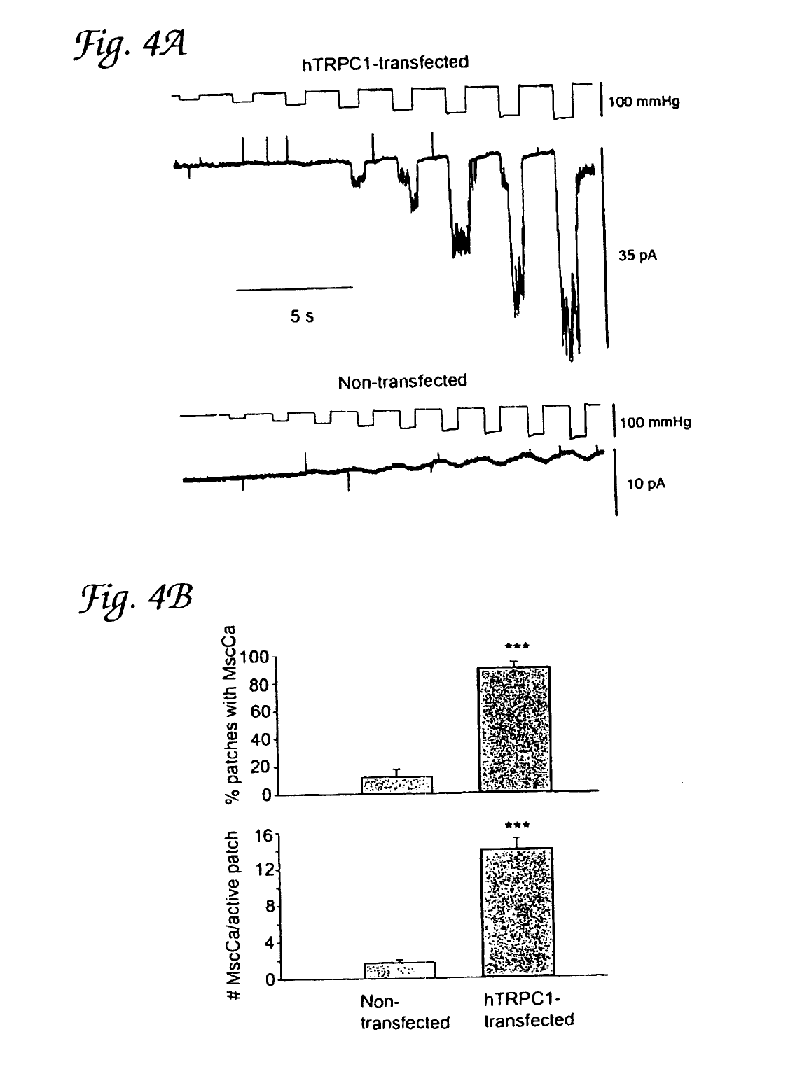

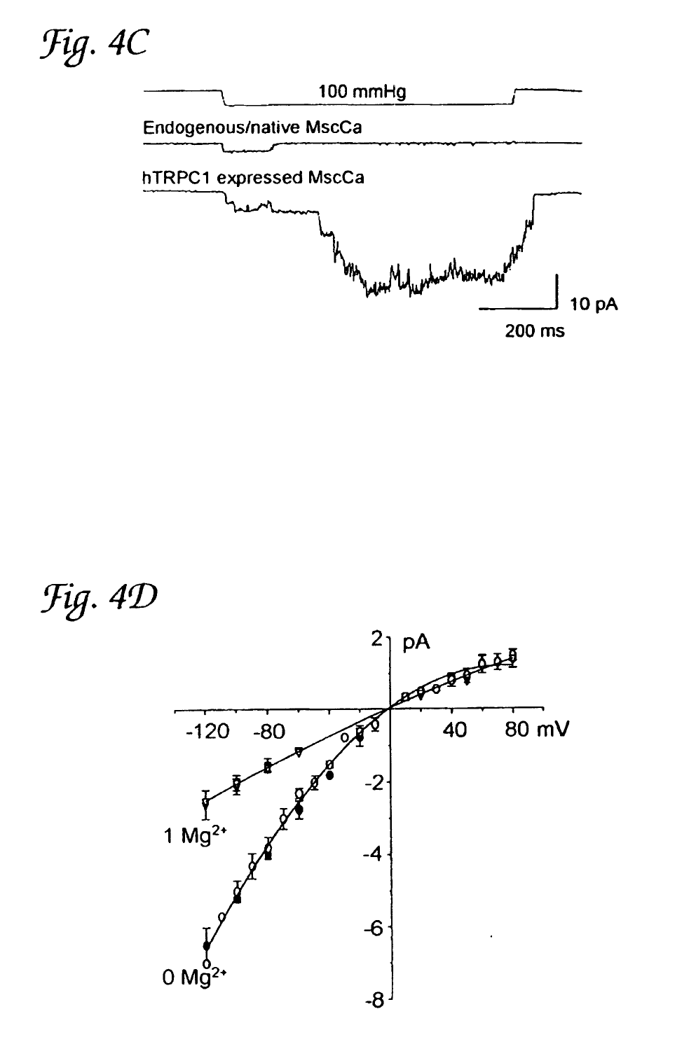

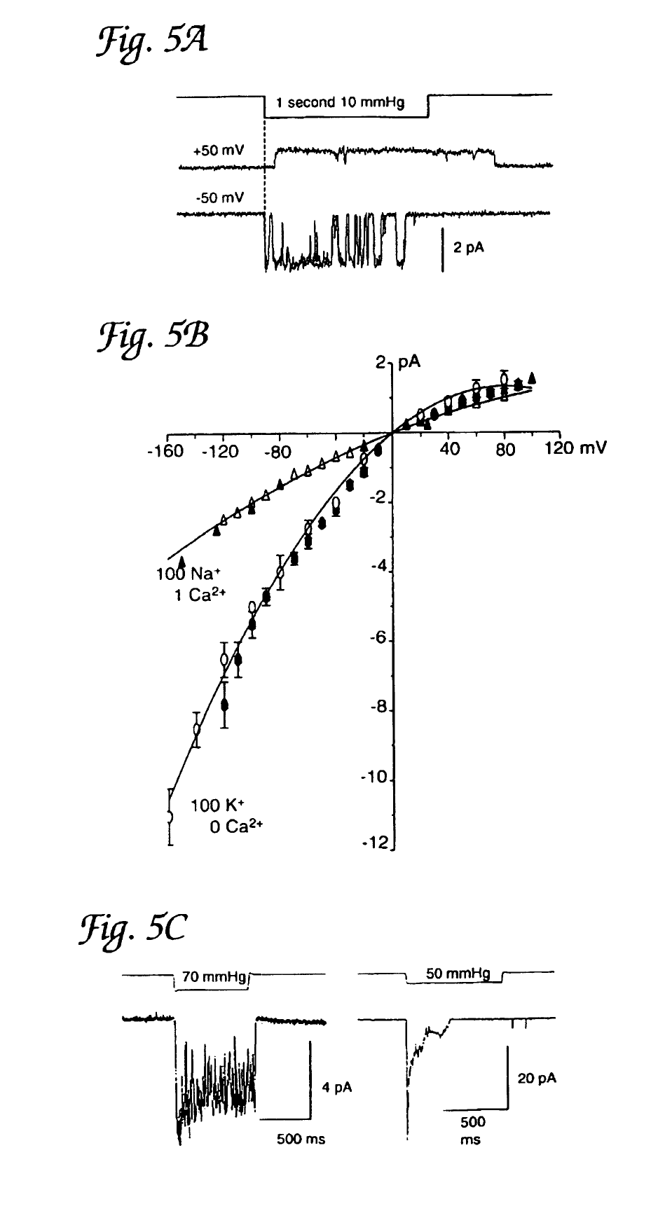

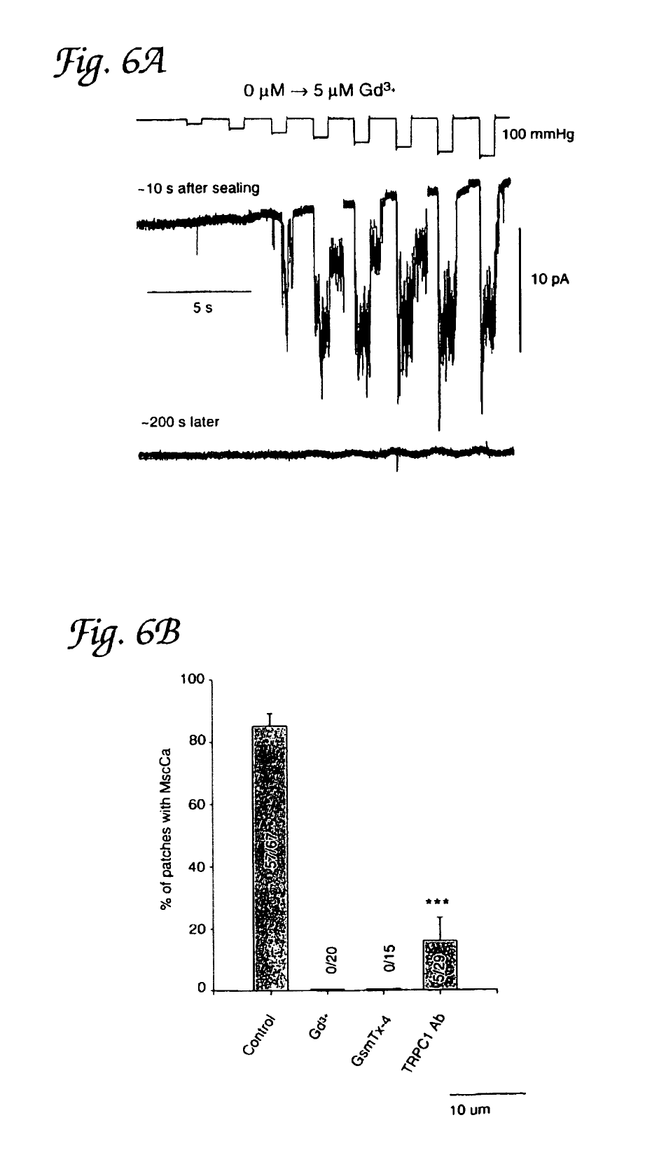

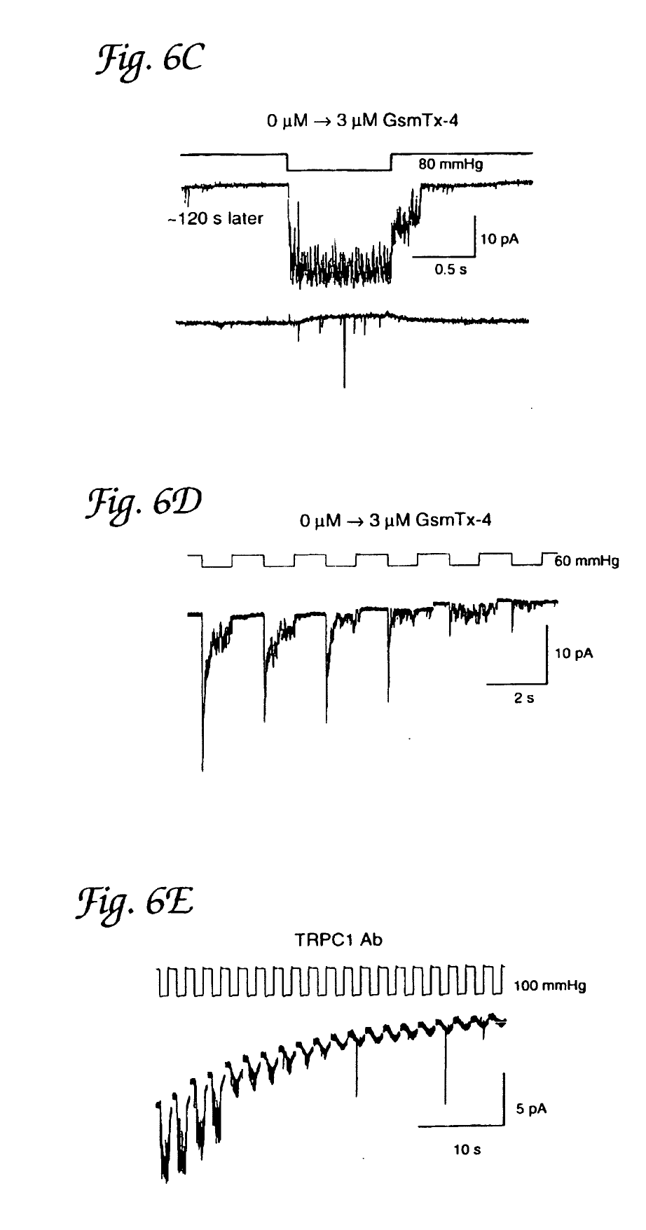

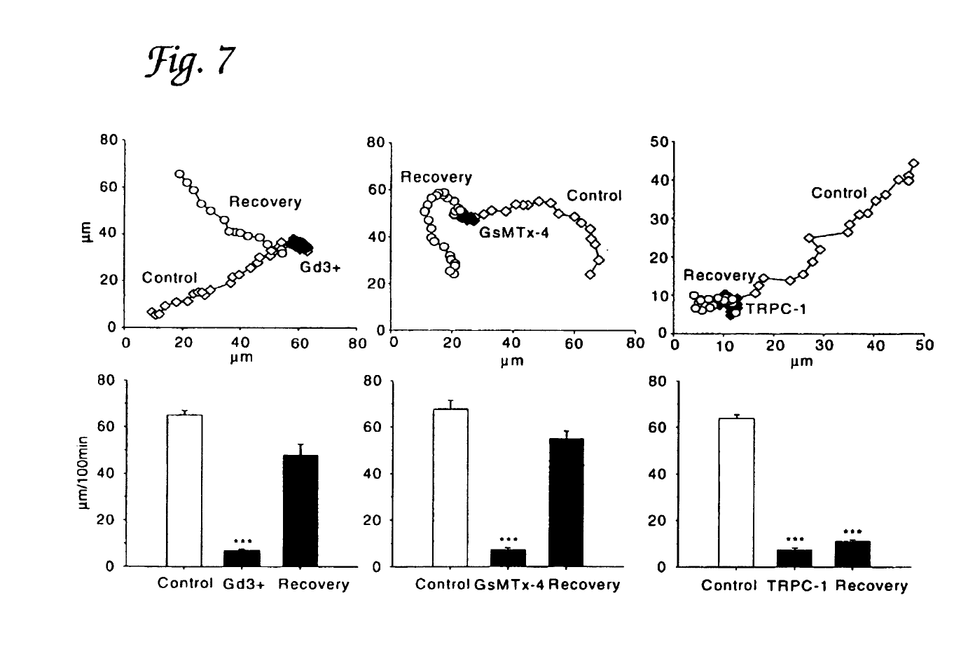

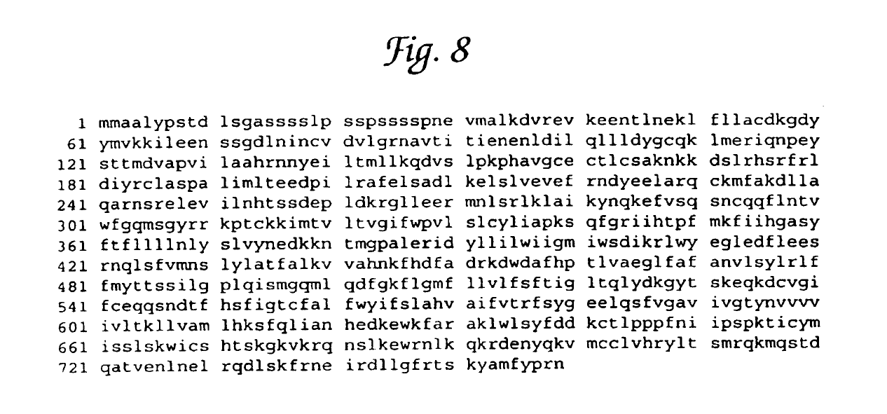

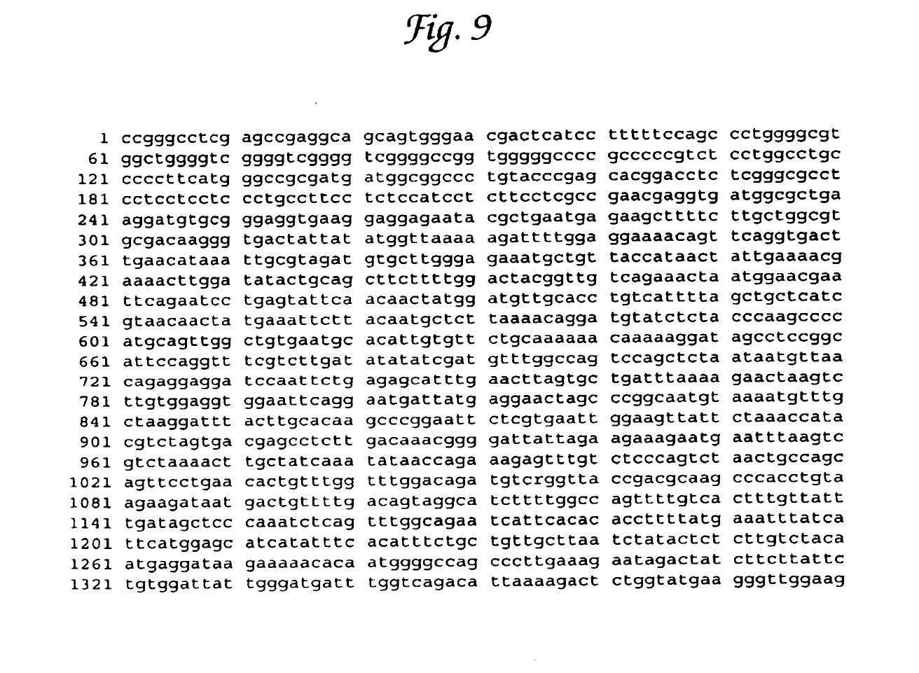

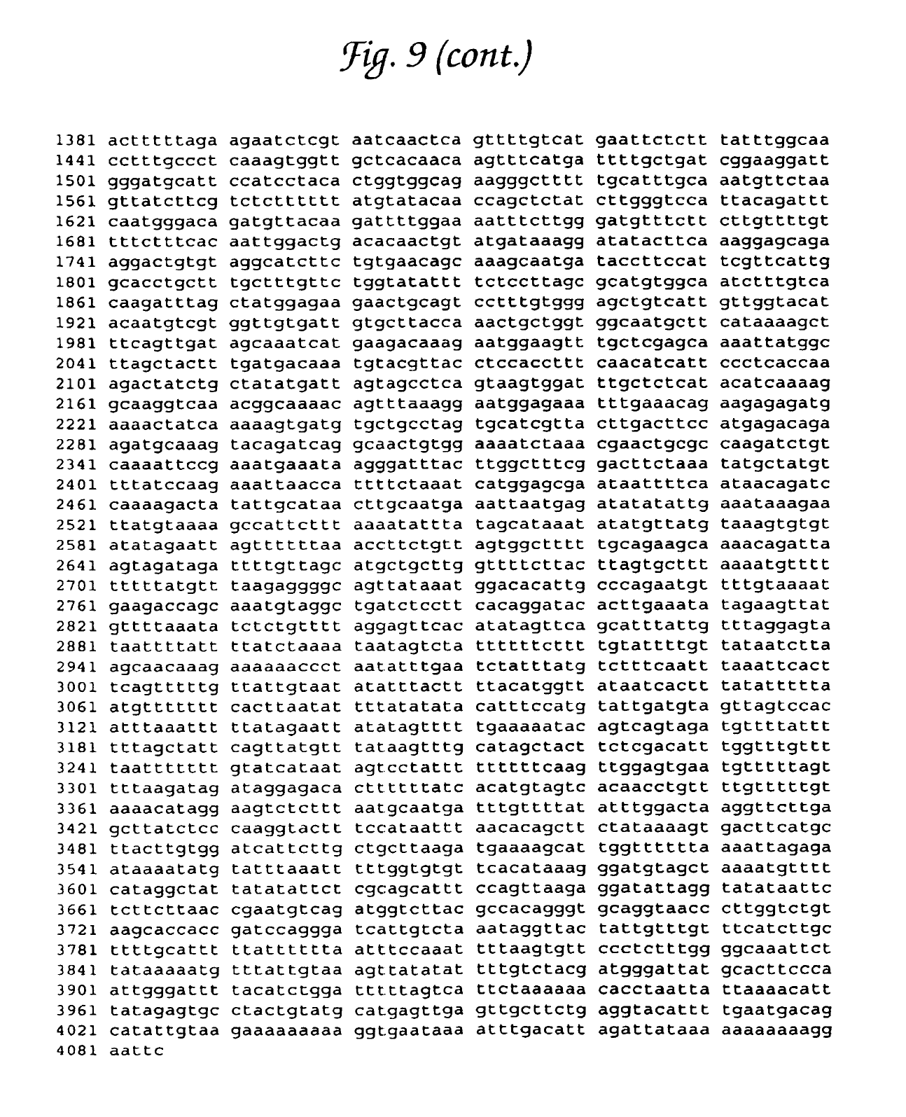

Because the hTRPC1 is highly homologous (84% identical and -90% similar in amino acids) to the Xenopus TRPCI and also results in expression of an apparent -80 kDa protein when expressed in oocytes (Brereton et al., Molec. Cell. Biochem. 214, 63-74 (2000)), the effects of heterologous expression of hTRPCI were tested on MscCa patch density in oocytes. Currents in response to increasing pressure steps applied to a cell-attached patch (pipette solution in mM: 100 mM KCI, 5 Hepes, 2 EGTA) on a control oocyte (water-injected) and an oocyte injected 4 days previously with hTRPCI transcripts were compared. The latter showed a several fold increase in the saturating currents (FIG. 2), yet the single channel currents were similar and displayed the same I-V relationship as the native MscCa (-50 pS at -50 mV, -10 pS at 50 mV). FIG. 3 shows the responses of another pair of patches to staircase increases in pressure in which saturating currents of --12 pA for the control (FIG. 3A) versus -170 pA for the hTRPCI-transfected oocyte patch were evoked (FIG. 3B, note the pressure for half maximum activation was -20 mmHg in each case) and translated into MscCa patch densities of 5 (7 0.8, 30 patches, 3 frogs) and 85 (98, 17, 15 patches, 2 frogs; FIG. 3C), respectively, assuming single channel currents of -2 pA. In comparison, injection of oocytes with antisense cRNA for hTRPCI reduced endogenous MscCa activity (1.25 0.32, 20 patches, 2 frogs, FIG. 3C) along with the 80 kDa band measured in Westerns. Gd³+, which blocks the oocyte MscCa (Wilkinson et al., J. Memb. Biol. 165, 161-174 (1998)) and also TRPC channels (Minke, B. & Cook, B., Physiol. Revs. 82, 429472 (2002)), was equally effective in blocking the native and the TRPC1-expressed MscCa. For the cell-attached patch recordings (FIGS. 3A & 3B) the pipette solution contained 100 mM KCI, 2 nM EGTA (KOH) and 5 mM Hepes (KOH) and the driving force was -20 mV (i.e., measured from the reversal potential of 0 mV of the MscCa channel currents). The two patch pipettes used for this comparison were pulled from the same capillary tube thereby ensuring identical tips.
To further test the idea that TRPCI forms the MscCa, several mammalian cell lines were screened as possible “nulls” for MscCa. However, of the seven cell lines tested, six (LMTK, Jurkat-T, HEK-293, BC3H-1, LNCaP and PC3) expressed relatively high MscCa activity (i.e., with 70-100% patches active), compared with one (CHO-K1) that expressed low MscCa activity (i.e., 10% active patches). Combined transmission and confocal fluorescence microscopy of hTRPCI-transfected and non-transfected CHO-KI cell colonies showed cells distinguished by their enhanced green fluorescence protein (eGFP) fluorescence. FIG. 4A shows cell-attached patch recordings from hTRPCI-transfected and non-transfected cells in response to increasing steps of pressure and indicate the increased MscCa expression in hTRCPCI-transfected (90% patches with 14 MscCa/active patch) versus non-transfected cells (10% patches with S 2 MscCa/active patch) (FIG. 4B). Recordings indicate that both the native CHO-KI and the hTRPC 1-expressed MscCa channels (FIG. 4C) display a similar l-V (current-voltage) relationship and Mg²+sensitivity as the frog oocyte MscCa (FIG. 4D). These results indicate that these MscCa properties have been highly conserved over several 100 million years of vertebrate evolution (e.g., Xenopus, hamster and human). Western analysis confirmed a significant increase in expression of TRPC1 in transfected versus non-transfected CHO-KI cells.
However, the same blot was negative when tested with an anti-TRPC4 antibody that detected a - I10 kDa band in rat brain, frog heart and oocyte membranes.
These results are consistent with the idea that TRPCI is an essential component of the MscCa. This represents a novel function for TRPC I.
Although previous studies indicate that TRPC), like the MscCa, is widely expressed in vertebrate cells, as described by Riccio et al. (Molec. Brain Res., 109, 95-104 (2002)), its function and physiological gating mechanism have been controversial. The identification of TRPC I as forming the MscCa indicates that TRPC1 acts in mechanotransduction.

### Example 3

### Voltage Characteristics of potential Mechanoselective Channel in Prostate Tumor Cells

To demonstrate that MscCa activity is required for prostate tumor cell migration, the voltage characteristics of membranes of the highly metastatic, motile cell line PC3, that is known to promote metastasis when introduced into nude mice, were evaluated. PC3 cell lines were grown in RPM1 1640 medium with 25 mM Hepes and glutamine, supplemented with 7% fetal calf serum, I mM sodium pyruvate, 4.5 g/L glucose, penicillin G (10,000 U/ml) and streptomycin sulfate (10,000 tg/ml) at 37° C. in a humidified 95% 02-5% C0₂ atmosphere. For patch-lamp studies, cells were seeded at low density on sterile coverslips placed in 33 mm culture dishes and tested during the next 2 - 5 days. Patch clamp recording was conducted as follows. Standard cell-attached patch-clamp recording was used to record single channel currents. The pipettes were made from thin-walled borosilicate glass (World Precision Instruments, Stevenage, UK) with an outside tip diameter of -2 Wm. Immediately prior to patch clamping, a cover slip was removed from the culture dish, glued with dots of silcone to a 35 mm culture, and placed in a microscope stage holder. Cells were visualized with an IM35 Zeiss microscope with fluorescence and Normaski optics. A pressure-clamp (McBride, D.W., Jr. & Hamill, O. P., PflLigers Archiv. 421, 606-612 (1992)) was used to apply pressure and suction protocols to achieve the initial tight seal and stimulate MS channels. To ensure minimal disruption of membrane-cytoskeleton interactions and retention of dynamic channel kinetics (adaptation), a gentle-sealing protocol was used (McBride, D.W., Jr. & Hamill, O.P., Pfliugers Archiv. 421, 606-612 (1992)); the suction during sealing was monitored continuously and did not exceed 10 mrnHg usually applied for less than 10 seconds to obtain a gentle seal.
The standard pipette solution contained in mM: 100 KCI, 2 EGTA (KOH), 5 Hepes (KOH) at pH 7.4.
FIG. 5A shows single channel currents activated by pressures steps applied to a cell-attached patch on a PC3 cell at -50 and +50 mV membrane potential. At the negative potential the channel showed repetitive openings and closings that was less evident at the positive potential. This voltage-dependent gating of the open channel is also a characteristic of the prototypical MscCa, native to the frog oocyte (Taglietti et al., J. Physiol., 407, 311 (1988)), identified as being formed by the transient receptor channel protein I (TRPCI). The current-voltage relationship of the prostate MscCa measured with a 100 mM K+, zero Ca²+in the pipette solution indicates an inwardly rectifying single channel conductance (-70 pS chord conductance at -100 mV and 20 pS at 50 mV) (Fig.
SB) that is essentially identical to that of the oocyte MscCa indicating a common pore structure. Likewise, replacement of K+by Na+did not significantly alter the I-V, whereas inclusion of 1 mM Ca²+reduced the inward current consistent with permeant ion block of the channel (FIG. 5A). Direct evidence indicating that the MscCa allows significant Ca²+influx under physiological conditions (i.e., with 1 mM external Ca²+) was that activation of MscCa inward currents resulted in a delayed activation of outward channels currents that were absent when either Ca²+was absent or 50 nM charybdotoxin (a specific Ca²+-activated K+channel blocker) was present in the pipette solution.
Ca²+influx through the MscCa is dependent upon the gating of the channel. The MscCa in PC3 cells displayed two distinct gating modes that were distinguished by their response to pressure steps. In the “sustained mode” (SM), the channels remained open for the duration of stimulation (Fig. SC), while in the “transient mode” (TM) the channels closed rapidly (cO.5 s) (FIG. 5C). The channels in the two gating modes showed the same single channel conductance but differed in their fragility in response to repetitive mechanical stimulation of the patch; whereas the SM was relatively robust, the TM was fragile and irreversibly disappeared with repetitive stimulation. Similar mechanical fragility in gating dynamics has been reported for the MscCa in other cells and may reflect the mechanical disruption of cytoskeleton-membrane interactions that convey mechanical forces to the MscCa protein (see Hamill et al., Proc. Nat. Acad. Sci. USA, 89, 7642 (1992)). This idea is supported by the observation that agents that target the cytoskeleton can also disrupt the TM of gating (see Suchyna et al., Phys. Biol. 1, 1 (2004)). The TM of gating in prostate tumor cells was shown to reflect stretch-induced inactivation of the channel rather than adaptation, because closed channels could only be reactivated after removal and reapplication of the pressure stimulus. The MscCa activity in the majority of patches (80%) formed on the highly motile, metastatic PC3 cells showed SM gating. As described in examples below, the different gating modes may contribute to the two kinetically distinct intracellular Ca²+fluctuations (i.e., transient and sustained) associated with PC3 cell movement. Interestingly, parallel studies of a non-motile, non-metastatic human prostate tumor cell line, LNCaP, indicate the large majority of patches (-90%) display the TM of gating and these cells show fast Ca²+₁ transients but do not develop sustained Ca²+J₁ gradients.

### Example 4

### Effect of Various Agents on Calcium Influx

The effects of gadolinium ion (Gd³+), GsmTx4 (Grammostola spatulata venom 4 kDa peptide), and anti-TRPCI antibody raised against a putative pore region of the TRPC I channel, as described by Wes et al. (Proc. Natl. Acad. Sci., 92, 9652 (1995)), on calcium influx and prostate tumor cell migration were studied. PC3 and LNCaP cell lines (ALTC CRL 1435 and ATCC CRL 1740 were grown in RPMI 1640 medium with 25 mM/Bepes and glutamine, supplemented with 7% fetal calf serum, 1 mM sodium pyruvate, 4.5 g/L glucose, penicillin G (10,000 U/mi) and streptomycin sulfate (10,000 mg/ml) at 37° C. in a humidified 95% 02-5% CO₂ atmosphere. For videomicrosopy and patch-clamp studies, cells were seeded at low density on sterile coverslips placed in 33 mm culture dishes and tested during the next 2 - 5 days.
To measure Ca²+block and perneation, I mnM Ca²+replaced the 2 mM EGTA. Gd³+was added to the pipette solution without EGTA present. To study different agents on the MscCa activity we used cell-attached patches rather than outside-out patches (OOP) because MscCa activity (i.e., TM gating) was not well retained in this configuration. In order to monitor MscCa patch density and gating mode before exposure to the agents the pipette tip was filled by capillary action - 300 gm from the orifice with agent-free pipette solution then the backfilled with the agent-containing solution. Using this procedure it was possible to monitor the MscCa activity before and after exposure to the agent. The standard bath solution contained in mM: 150 NaCl, 2.5 KCI, 2 CaCI₂, I MgCI₂ and 10 Hepes (NaOH) at pH 7.4. Patch currents were filtered at 500 Hz with using an 8-pole Bessel filter and digitized at 1 kHz on an IBM clone using pCLAMP (Axon Instruments) acquisition and analysis software packages.
FIG. 6A shows cell-attached patch currents measured in response to increasing steps of pressure applied immediately after forming the tight seal on the cell and then again -3 minutes later. This time interval is sufficient to allow Gd³+, which was included in the back solution (at 5 gM) to diffuse into the pipette tip and block the channel. In other experiments, in which Gd³+was also included the pipette-tip solution, all MscCa activity was abolished (20 out of 20 patches, FIG. 6B). GsmTx-4, a tarantula venom peptide, which is the most specific MscCa channel blocker reported to date (Suchyna et al., J. Gen. Physiol., I 15, 583 (2001)), also blocked MscCa activity in PC3 cells. FIGS. 6 B-D indicates that 3 FM GsmTx-4 was effective in blocking the MscCa activity when either in the SM (FIG. 6C) or TM (FIG. 6D) gating mode.
The third agent tested was an anti-TRPC I antibody (Ab) raised against the putative pore region of the TRPC I channel. Anti-TRPC I antibody was identified in Westerns of the PC3 cell membranes as an 80 kDa protein, as described below. This is consistent with previous studies in oocytes and CHO cells using the same antibody. Moreover, confocal immunofluorescence microscopy of living PC3 cells, indicated that the TRPCl protein was present on the surface of the cell and was concentrated predominately in the cell rear with particularly high aggregates in the extended tether. Cell-attached patch recordings with the anti-TRPCI Ab (20 gg/ml) included in the pipette decreased but did not abolish MscCa activity (FIGS. 6E).
Immunofluorescence and confocal microscopy were carried out as follows. Cultured cells on cover slips were incubated for 30 minutes, 37° C. in mammalian Ringer's solution in the presence of anti-TRPC-1 Ab (10 tg/mi) raised to the external pore region of TRPCI. After washing three times with Ringer's solution, cells were incubated with the secondary antibody (2.5 jg/ml, goat anti rabbit ALEXA fluor 568) during 30 minutes at room temperature with very gentle shaking. The incubated cells were then washed several times and fixed in fixative solution (2.5% formaldehyde, 0.1% glutaraldehyde, 0.03% picric acid, 0.03% CaCI₂ and 0.05M cacodylate buffer pH 7.4). Control experiments were performed by single incubation with the secondary antibody. A Zeiss LSM 510 META confocal system configured on an Axiovert 200M inverted microscope was used to acquire the images at 543 nm excitation (green HeNe laser).
For Western Blot analysis, cell lysates were collected in ice-cold buffer (10 mM Tris, 150 mM NaCl, 5mM E)TA, IM KI, I mM phenylmethanesulfonyl fluoride (PMSF), 2.5 Al/ml protease inhibitor cocktail, Sigma, #P 8340), and 1.5% OG), vigorously shaken at 4° C. for 15 minutes and centrifuged at 14,000 RPM, 4° C, 30 min. The supernatants were stored at 80° C. Protein concentration was measured using bicinchonic acid (BCA) assay (Pierce BIO-RAD, Rockford, IL). Protein samples were resolved in an 8% SDS/PAGE gel, transferred to a PVDF membrane and developed by enhanced chemijuminescence. Blot incubation with primary antibody was carried out overnight in the cold room by gentle shaking. An anti-TRPCI Ab was generated against the Tle3 epitope (CVGIFCEQQSND) (SEQ ID NO: 5) that lies between the S5 and S6 membrane spanning domains, the proposed to pore region of the channel (Bobanovic et al., 1999). Preliminary results indicated the specificity of this anti-TRPC- I Ab to the 80 kDa protein compared to a commercial anti-TRPCI Ab (Alomone Labs, lot # AN-02) raised against the peptide QLYDK GYTSK EQKDC (amino acid residues 557-571 of hTRPCI, accession # P48995) (SEQ ID NO: 6). The specificity of the antibody protein binding was tested using the antigenic peptide.

### Example 5

### Effect of Various Agents on Tumor Cell Mipration

The effect of the three anti-MscCa agents on PC3 cell migration was tested using time-lapse videomicroscopy to record the trajectory of the migrating prostate tumor cells. Selected video frames were prepared at 15 minute intervals, showing a cluster of 7 PC3 cells as they migrate out from the cluster. FIG. 7 (top panels) shows selected PC3 cell trajectories for one hour intervals before, during and after exposure to Gd³+(5 ttM), GsMTx-4 (3 μM) and anti-TRPC I Ab (10 ji/ml); the lower panels show histograms of rates of migration. All three agents blocked cell migration. However, whereas the block by Gd³+and GsMTX-4 block was reversible with washout, the Ab block did not show reversal even after 1 hour of washout (FIG. 7). Control experiments preincubating the Ab with the control peptide indicated that the block was specific.

Because Ca²+influx and Ca²+)influctuations have been shown to be important in regulating migration in a wide variety of cells-types including cancer cells (Huang et al., Cancer Res., 64, 2482 (2004)) the effects of anti-MscCa agents on [Ca²+]i fluctuations were tested in migrating PC3 cells. Two distinct types of [Ca²+]i fluctuations were observed; a sustained Ca²+)₁ that increased from the front of the cell to the rear and was maintained for the duration of the locomotion cycle (up to 3 hours) measured from tether formation to tether release; and a fast transient [Ca2+i wave that spread rapidly through out the cell and lasted <1 minute (the time interval between frames) with an average frequency of 6 waves in 30 minutes. Both sustained and transients [Ca2+]i fluctuations were associated with moving cells. All three anti-MscCa agents described above, abolished both forms of [Ca²+]i oscillations at the same concentrations that blocked/reduced MscCa activity and PC3 cell migration (9 cells, 3 experiments). Again, whereas with Gd and GsmTx4 the block was reversible, there was no recovery after the anti-TRPCI Ab washout.

Time-lapse videomicroscopy and Ca 2-imaging were conducted as follows. Cell migration was monitored by time-lapse videomicroscopy using Nomarski optics with an Epifluorescent microscope (TE 200). The time-lapse images were acquired and saved at 5 minute intervals using Metafluor software. Calcium imaging assays were carried out on cells previously loaded with Fura-2 AM (5 tm for 30 minutes). Time-lapse fluorescence images were consecutively acquired at 340 and 380 wavelengths and transmission (DIC) at intervals of I minute using Metafluor. Data analysis was carried out using Metamorph (Version 6. 1) and Excel] 2000. Conversion of pixels to gm based on a calibrated slide to represent the cell trajectories in the field.

### Examle6

### Gene Silencing Effect on Calcium Influx and Tumor Cell Migration

PC3 transfected with siRNA for TRPCI also failed to migrate whereas cells transfected with scrambled siRNA expressed normal migration. The day before transfection cells were seeded at 50-60% density on sterile coverslips in a 6 well plate in culture medium without antibiotic. Transfection of 100 nM of double stranded siRNA Cy3 labeled per well (Ambion #731 1 and negative control, Ambion, Austin, TX was carried using Lipofectamine 2000 (Invitrogen, Carlsbad, CA) according to the manufacturer's instructions for siRNA transfections. Cells were stored in the incubator after the addition of the complexes and 7% FCS medium without antibiotic was added after the first 6 hours of transfection. The next day the supernatant was removed and normal culture medium replaced. Cells were tested by patch clamp and videomicroscopy during the next 48-72 hours, using the patch clamp and videomicroscopy methods described above in examples 4 and 5. TRPCI siRNA transfection blocked PC3 cell migration and Ca²+fluctuations and also reduced MscCa expression measured with patch clamp.
The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified

What is claimed is:

1. A method for identifying an agent that decreases activity of a mechanosensitive Ca²⁺-permeable (MscCa) channel comprising:
contacting a cell expressing an MscCa channel with a candidate agent to yield a treated cell; and
comparing the activity of an MscCa channel of the treated cell with the activity of an MscCa channel of a control cell not contacted with the candidate agent, wherein decreased activity of an MscCa channel of the treated cell indicates the candidate agent decreases the activity of an MscCa channel.

2. The method of claim 1 wherein the MscCa channel comprises a polypetide comprising an amino acid sequence of at least 90% identity to SEQ ID NO: 2, wherein the polypeptide has MscCa activity.

3. The method of claim 1 wherein the MscCa channel comprises a polypeptide comprising SEQ ID NO: 2.

4. The method of claim 1 wherein the cell is a tumor cell.

5. The method of claim 1 wherein the cell is a human prostate tumor cell line.

6. The method of claim 5 wherein the human prostate tumor cell line is ATCC CRL-1435.

7. The method of claim 1 wherein the cell is a motile cell, and wherein the treated motile cell has decreased motility compared to the control cell.

8. The method of claim 1 wherein the cell is an invasive cell, and wherein the treated invasive cell has decreased invasiveness compared to the control cell.

9. The method of claim 1 wherein the treated cell has decreased proliferation compared to the control cell.

10. The method of claim 1 wherein the treated cell has increased apoptosis compared to the control cell.

11. An agent identified by the method of claim 1.

12. A method for identifying an agent that decreases a phenotype of a cell comprising:
contacting a cell expressing an MscCa channel with a candidate agent to yield a treated cell; and
comparing the phenotype of the treated cell with the phenotype of a control cell not contacted with the candidate agent, wherein the phenotype is selected from the group of motility, invasiveness, proliferation, and a combination thereof, and wherein a decreased phenotype for the treated cell indicates the candidate agent decrease the phenotype.

13. The method of claim 12 wherein the candidate agent causes activity of an MscCa channel of the treated cell to decrease.

14. The method of claim 12 wherein the MscCa channel comprises a polypeptide comprising an amino acid sequence of at least 90% identity to SEQ ID NO: 2, wherein the polypeptides has MscCa activity.

15. The method of claim 14 wherein the MscCa channel comprises a polypeptide comprising SEQ ID NO: 2.

16. The method of claim 12 wherein the cell is a tumor cell.

17. The method of claim 12 wherein the cell is a human prostate tumor cell line.

18. The method of claim 17 wherein the human prostate tumor cell line is ATCC CRL-1435.

19. An agent identifies by the method of claim 12.

20. A method for treating cancer comprising:
administering to a subject having cancer an effective amount of a composition comprising an agent that decreases activity of a mechanosensitive ion channel present on a cancer cell, wherein a symptom of the cancer is decreased.

21. A method for decreasing metastasis of a cancer cell comprising:
administering to a subject at risk of developing cancer an effective amount mechanosensitive ion channel.

22. A method for decreasing a symptom associated with cancer comprising:
administering to a subject having cancer an effective amount of a composition comprising an agent that decreases activity of a mechanosensitive ion channel.

23. The method of claim 20, 21, or 22 wherein the mechanosensitive ion channel is a mechanosensitive Ca²⁺-permeable (MscCa) channel.

24. The method of claim 20, 21, or 22 wherein the agent is a polypetide comprising an amino acid sequence comprising at least 90% identity to SEQ ID NO: 1 or to SEQ ID NO: 7.

25. The method of claim 24 wherein the agent is a polypeptide comprising SEQ ID NO: or to SEQ ID NO: 7.

26. The method of claim 24 wherein the agent is a polypeptide comprising SEQ ID NO: 1 or SEQ ID NO: 7.

27. The method of claim 26 wherein the antibody binds to an epitope present on SEQ ID NO: 5 or SEQ ID NO: 6.

28. The method of claim 23 wherein the MscCa channel comprises an MscCa polypeptide, and wherein the agent is a polynucleotide that decreases expression of the MscCa polypeptide.

29. The method of claim 20, 21, or 22 wherein the cancer is prostate cancer, breast cancer, colon cancer, lung cancer, bladder cancer, ovary cancer, pancreas cancer, or skin cancer.

30. The method of claim 23 wherein the agent decreases activity of an MscCa channel comprising a polypeptide comprising SEQ ID NO: 2.

31. A method for inhibiting expression of an MscCa polypeptide comprising:
administering into a cell an effective amount of an RNA polynucleotide, wherein the polynucleotide comprises a sense strand and an antisense strand, wherein the sense strand comprises a nucleotide sequence of between 16 and 30 nucleotides, wherein the nucleotide sequence is substantially identical to consecutive nucleotides of an mRNA encoding a polypeptide of SEQ ID NO: 2, and wherein the cell comprising the RNA polynucleotide has decreased MscCa activity, decreased motility, decrease invasiveness, or a combination thereof, when compared to a control cell that does not comprise the RNS polynucleotide.

32. A method for treating cancer comprising:
administering to a subject having cancer an effective amount of an RNA polynucleotide, wherein the polynucleotide comprises a sense strand and an antisense strand, wherein the sense strand comprise a nucleotide sequence of between 16 and 30 nucleotides, wherein the nucleotide sequence is substantially identical to consecutive nucleotides of an mRNA encoding a polypeptide of SEQ ID NO: 2, and wherein a symptom of the cancer is decreased.

33. A method for decreasing metastasis of a cancer cell comprising:
administering to a subject at risk of developing cancer an effective amount of an RNA polynucleotide, wherein the polynucleotide comprises a sense strand and an antisense strand, wherein the sense strand comprises a nucleotide sequence of between 16 and 30 nucleotides, and wherein the nucleotide sequence is substantially identical to consecutive nucleotides of an mRNA encoding a polypeptide of SEQ ID NO: 2.

34. A method for decreasing a symptom associated with cancer comprising:
administering to a subject having cancer an effective amount of an RNA polynucleotide, wherein the polynucleotide comprises a sense strand and an antisense strand, wherein the sense strand comprises a nucleotide sequence of between 16 and 30 nucleotides, wherein the nucleotide sequence is substantially identical to consecutive nucleotides of an mRNA encoding a polypeptide of SEQ ID NO: 2.
